(19) United States
(12) Patent Application Publication
(10) Pub. No.: US 2015/0147292 A1
(43) Pub. Date: May 28, 2015

(54) NOVEL SURFACE MARKERS FOR ADIPOSE TISSUE

(71) Applicants: C. Ronald Kahn, West Newton, MA (US); Siegfried Ussar, Munich (DE)

(72) Inventors: C. Ronald Kahn, West Newton, MA (US); Siegfried Ussar, Munich (DE)

(73) Assignee: Joslin Diabetes Center, Inc., Boston, MA (US)

(21) Appl. No.: 14/344,936

(22) PCT Filed: Sep. 14, 2012
(86) PCT No.: PCT/US12/55479

§ 371 Date: Feb. 10, 2015

Related U.S. Application Data
(60) Provisional application No. 61/535,194, filed on Sep. 15, 2011.

(51) Int. Cl. A61K 47/48; A61K 38/20; A61K 38/28; A61K 31/426; A61K 38/22; A61K 38/17; A61K 38/19; A61K 38/18

(52) U.S. Cl. CPC A61K 47/48561; A61K 38/191; A61K 38/204; A61K 47/48092; A61K 38/1825; A61K 38/1875; A61K 31/426; A61K 38/22; A61K 38/1709; A61K 38/28
USPC 424/85.2; 530/391.7; 424/178.1; 530/399; 530/351; 536/23.1; 536/24.5; 514/44 R; 514/44 A; 424/85.1

(57) ABSTRACT

The present invention is based in part on the discovery of brown and white fat cell specific surface markers. It has been found that the small amino acid transporter Slca10/Asc1 is a specific surface marker for white adipocytes and that the ligand-gated ion channel P2X5 and the small amino acid transporter Slc36a2 are specific surface markers for brown adipocytes. Having identified these specific white and brown cell surface markers, the present invention provides compositions and methods suitable for the targeting of any number of agents to a white or brown adipose tissue and the identification and isolation of white or brown adipocytes for any number of uses including therapeutic, screening and diagnostic purposes.

### BACKGROUND

The pandemics of obesity, diabetes and metabolic syndrome are on the rise, in part caused by excessive energy intake and too little energy expenditure. While this basic thermodynamic principle seems obvious, its translation into effective treatments for these disorders is still lacking. Adipose tissues are key in the fight against obesity. In the past, most attention has been focused on white adipose tissue, as a site of energy storage. Over the past decade, however, great advances have been made in understanding how white adipose tissue, via the secretion of adipokines, can have systemic effects and regulate appetite and whole body energy balance (Trujillo and Scherer, Endocr. Rev. 27: 762-778 (2006; Ahima, Trends Endocrinol Metab 16:307-313 (2005)). White adipocytes in different depots impart different risks of metabolic disease and have different patterns of adipokine secretion, a property believed to be at least in part due to developmental differences between white adipocytes in different depots (Gesta, et al., Proc. Natl. Acad. Sci. 103:6676-6681 (2006); Tchkonia, et al., Am J Physiol Endocrinol Metab 292:E298-E307 (2007)). In addition, in obesity, adipose tissue is a site of chronic inflammation which contributes to systemic insulin resistance through secretion of inflammatory cytokines (Shoelson, et al., J. Clin Invest 116:1793-1801 (2006)). Previously it has been shown that subcutaneous fat transplanted into the visceral cavity has beneficial effects on the metabolism in mice albeit its changed anatomical location (Tran, et al., Cell Metab 7(5): 410-420 (2008)).

More recently, brown adipose tissue (BAT), specialized on energy expenditure, has gained more attention (Tseng et al., Nat Rev Drug Discov. 9:465-482 (2010)). BAT is essential for non-shivering and diet induced thermogenesis. Its function is mainly mediated by mitochondrial uncoupling via uncoupling protein 1 (UCP1), a protein predominantly expressed in brown adipocytes (Golozoubova, et al., FASEB J 15:2048-2050 (2001)). Recent studies suggest that at least two distinct pools of brown adipocytes also exist that appear to have different developmental origins (Atit, et al., Dev Biol 296: 164-176 (2006); Petrovic, et al., J Biol Chem 285:7153-7164 (2010); Seale et al., Nature 454: 961-967 (2008); Tseng et al., Nat Rev Drug Discov. 9:465-482 (2010)). In rodents and human infants, the most distinct pool of brown adipocytes forms an interscapular BAT. In adult humans, as well as rodents, there are additional sites where brown adipocytes reside. Indeed, in adult humans, the major depots of functionally active brown fat have been located in the neck and supraclavicular region, as well as in paraspinal areas (Cypess, et al., Curr Opin Pediatr 22:478-484 (2009)). Histologically, these depots consist of a mixture of brown and white adipocytes. However, there is still little known about the exact control of brown versus white preadipocyte commitment; the control of relative amounts and functional differences between white and brown adipocytes in different depots; the heterogeneity and intrinsic differences of adipocytes in different depots and the exact pathways of fat cell development.

A major limitation for the advancement in these areas is the identification, tracking and targeting of these different types of white and brown adipocytes in vivo. Currently identification and classification relies mainly on the differences in density of adipose tissue on different scanning techniques (CT, MRI, etc.) and their anatomical location (visceral vs. subcutaneous). Histological differences are limited to those situations where surgical biopsies can be obtained, and this is often difficult for many areas deep in the body. For the differentiation of brown vs. white fat, there has been some success using PET/CT scanning which takes advantages of the metabolic differences between these types of fat (brown is more metabolically active that white). However these criteria fail to fully describe and identify the different adipocytes, which may occur in mixed depots. Furthermore, the metabolic activity of brown adipocytes, currently the only way to identify this tissue in humans, is influenced by various environmental factors, like ambient temperature, diet and drugs (Cypess, et al., Curr Opin Pediatr 22:478-484 (2009)). While the use of transgenic mouse models and cell lines has provided a tool for basic research, this approach is not applicable to humans. Therefore what is needed in the art are agents and methods suitable to identify brown and white fat cell specific surface markers, to provide a novel tool to identify specific adipocyte pools and target them for therapy in vivo.

### SUMMARY OF THE INVENTION

The present invention is based in part on the discovery of brown and white fat cell specific surface markers. In particular, it has been found that the small amino acid transporter Slca10/Asc1 is a specific surface marker for white adipocytes and that the ligand-gated ion channel P2X5 and the small amino acid transporter Slc36a2 are specific surface marker for brown adipocytes. Having identified these specific white and brown cell surface markers it is now possible to target any number of agents to a white or brown adipose tissue or white or brown adipocytes for any number of uses including therapeutic, screening and diagnostic purposes.

Accordingly, in one aspect, the invention provides compositions including a P2X5 targeting agent or a Slc36a2 targeting agent coupled to a BAT agent that modifies BAT activity. In one embodiment, the P2X5 targeting agent or Slc36a2 targeting agent is an antibody, antibody fragment or aptamer. In a further embodiment, the BAT agent increases BAT mass, promotes BAT activity or both. In another embodiment, the BAT agent decreases BAT mass, inhibits BAT activity or both. In one aspect the BAT agent can be a bone morphogenetic protein (BMP) polypeptide or fragment thereof, e.g., BMP-2, BMP-4, BMP-6, BMP-7; a fibroblast growth factor, e.g., FGF-6, FGF-9; a small molecule ligand, e.g., thiazolidinedione and an siRNAs.

In one embodiment, the invention provides for methods of targeting an agent to a brown adipose tissue or brown adipocyte in a subject by administering a therapeutically effective amount of the agent to be targeted to a brown adipose tissue or brown adipocyte whereby the agent is coupled to a P2X5 targeting agent or a Slc36a2 targeting agent. The agent is preferably a BAT agent that modifies BAT activity and preferably a BAT agent that increases BAT mass, promotes BAT activity or both. Alternatively the BAT agent decreases BAT mass, inhibits BAT activity or both. The P2X5 targeting agent is preferably an antibody, antibody fragment or aptamer to P2X5. The Slc36a2 targeting agent is preferably an antibody, antibody fragment or aptamer to Slc36a2.

In another embodiment, the invention provides methods for detecting brown adipose tissue or brown adipocyte in a subject including the steps of administering to the subject an effective amount of a P2X5 targeting agent or a Slc36a2 targeting agent coupled to a detecting agent, and measuring the amount of detecting agent on the brown adipocytes.

In another embodiment, the invention provides a method of sorting brown adipocytes from a cell sample where the cell sample contains a mixed population of cells. The method includes the steps of providing a cell sample suspected of containing brown adipocytes and a probe with specificity to P2X5 or Slc36a2, contacting the cells with the probe, then identifying brown adipocytes by detecting the probe; and, isolating at least a portion of the identified brown adipocytes from the tissue sample.

In another aspect the invention provides compositions including a Slc7a10 targeting agent coupled to a WAT agent that modifies WAT activity. In one embodiment, the Slc7a10 targeting agent is an antibody, antibody fragment or aptamer. In a further embodiment, the WAT agent increases WAT mass, promotes WAT activity or both. In another embodiment, the WAT agent decreases WAT mass, inhibits WAT activity or both. In one aspect the WAT agent is a growth factors, hormones (e.g. insulin, BMPs, FGFs), small molecule ligands (thiazolidinediones), small interfering RNAs targeting e.g. resistin, RBP4, TNFa, IL-6), toxins).

In one embodiment, the invention provides for methods of targeting an agent to a white adipose tissue or white adipocyte in a subject by administering a therapeutically effective amount of the agent to be targeted to a brown adipose tissue or brown adipocyte whereby the agent is coupled to a Slc7a10 targeting agent. The agent is preferably a WAT agent that modifies WAT activity and preferably a WAT agent that increases or decrease lipid storage and or increases or decreases adipokine secretion. The Slc7a10 targeting agent is preferably an antibody, antibody fragment or aptamer to Slc7a10.

In another embodiment, the invention provides methods for detecting white adipose tissue or white adipocyte in a subject including the steps of administering to the subject an effective amount of a Slc7a10 targeting agent coupled to a detecting agent, and measuring the amount of detecting agent on the white adipocytes.

In another embodiment, the invention provides a method of sorting white adipocytes from a cell sample where the cell sample contains a mixed population of cells. The method includes the steps of providing a cell sample suspected of containing white adipocytes and a probe with specificity to Slc7a10, contacting the cells with the probe, then identifying white adipocytes by detecting the probe; and, isolating at least a portion of the identified white adipocytes from the tissue sample.

### BRIEF DESCRIPTION OF THE FIGURES

FIG. 1 shows (A) a schematic diagram of the Symatlas and BioGPS (www.biopgs.gnf.org) database search to identify genes with correlated expression profile to known adipocyte markers. (B) BioGPS diagram showing affymetrix expression data for murine adiponectin. (C) BioGPS diagram showing affymetrix expression data for murine neuregulin 4.

FIG. 2 shows (A) a Real Time PCR from twenty tissues of seven weeks of C57/BL6 mice (n=1). The blue bars indicate Slc7a10 expression normalized to the “TATA box binding protein” (TBP). The green bars show Slc7a10 expression normalized to 18s rRNA. (B) Real Time PCR from freshly isolated adipocytes and their corresponding stromal vascular fraction (SVF) from different fat depots (n=4).

FIG. 3 shows Western Blots for Slc7a10 from eighteen different tissues of PBS perfused seven weeks old C57/BL-6 mice. (A) Poinceau staining and α-actin were used as loading controls. (B) Additional signals at various molecular weights can be detected with the Slc7a10 antibody, indicating either cross-reactivity of the antibody or additional splice variants of Slc7a10.

FIG. 4 shows Real Time PCR for Slc7a10 from different white fat depots in the kidney and liver of ob/ob and control (ob/+) mice. Slc7a10/Asc-1 expression was shown normalized to TBP (n=5).

FIG. 5 shows (A) Real Time PCR of Slc7a10 from 3T3-L1 during an eight day time course of differentiation. AP2 is shown as a marker for mature adipocytes. Slc7a10/Asc-1 and AP2 expression are shown normalized to TBP (n=9). (B) Real Time PCR from in vitro differentiated primary preadipocytes from perigonadal and flank adipose tissue. The term “non-diff. adipocytes” indicates cells treated with the differentiation cocktail that did not show induction of differentiation markers or lipid accumulation (n=2). Slc7a10/Asc-1 and AP2 expression are shown normalized to TBP.

FIG. 6 shows Real Time PCR for Slc7a10, PPARγ and Adipsin from subcutaneous, mesenteric and omental primary human preadipocytes in vitro differentiated for seven or eight days (n=2). The “10 day” bar is set forth immediately to the right of the “7 day” bar in each instance.

FIG. 7 shows (A) Real Time PCR from twenty tissues recovered from seven week old C57/BL6 mice (n=1). The blue bars indicate P2X5 expression normalized to TBP. Green bars show P2X5 expression normalized to 18s rRNA. (B) Real Time PCR from freshly isolated adipocytes and their corresponding SVF from different fat depots (n=4).

FIG. 8 shows Western Blots for P2X5 from eighteen different PBS perfused tissues from seven week old C57/BL-6 mice. (A) Multiple signals at various molecular weights are detected with the P2X5 antibody. (B) Poinceau staining and α-actin were used as loading controls.

FIG. 9 shows (A) Real Time PCR of P2X5 from 3T3-L1 during an eight day time course of differentiation. P2X5 expression is shown normalized to TBP (n=3). (B) Real Time PCR from undifferentiated (day 0) and differentiated (day 8) murine brown preadipocytes (n=3). P2X5 expression is shown normalized to TBP.

FIG. 10 shows Real Time PCR from twenty tissues recovered from seven week old C57/BL6 mice for Nrg4 and six genes of transmembrane proteins with correlated expression profile (n=1). The blue bars indicate gene expression normalized to TBP. Green bars show gene expression normalized to 18s rRNA.

FIG. 11 shows Real Time PCR from freshly isolated adipocytes and their corresponding SVF from different fat depots (n=4).

FIG. 12 shows Real Time PCR for GPR119 and Slc36a2 from twenty tissues recovered from seven week old C57/BL6 mice (n=1) and freshly isolated adipocytes and their corresponding SVF from different fat depots (n=4).

FIG. 13 shows Real Time PCR for CD300LG, FZD4 and Tspan18 from twenty tissues recovered from seven week from C57/BL6 mice (n=1) and freshly isolated adipocytes and their corresponding SVF from different fat depots (n=4).

### DETAILED DESCRIPTION OF THE INVENTION

Using a combination of in silico, in vitro and in vivo approaches, we have identified Slc7a10 as a white adipocyte surface marker and P2X5 and Slc36a as surface markers for brown adipocytes. Importantly both surface proteins not only show a greatly increased expression in either white adipose tissue (WAT) or brown adipose tissue (BAT) but are also specific to adipocytes within the adipose tissue. These surface proteins provide a novel tool to selectively and non-invasively access these cells for therapeutic, diagnostic and or screening purposes. In addition these surface proteins can be used to detect BAT and WAT within living organisms and to deliver drugs specifically to WAT or BAT adipocytes through the use of any number of agent including but not limited to chimeric proteins, aptamers or small molecule conjugates. This would provide the ability to lower side effects of drugs due to effects on off target tissues. Finally the use of these markers allows for the sorting of brown and white adipocytes from whole tissues.

### I. DEFINITIONS

“Adipose cells” or adipocytes, also known as fat cells, are the cells that primarily compose adipose tissue, specialized in storing energy as fat. There are two types of adipose tissue, white adipose tissue (WAT) and brown adipose tissue (BAT), which are also known as white fat and brown fat, respectively, and comprise two types of fat cells.

“White fat cells” or monovacuolar cells contain a large lipid droplet surrounded by a layer of cytoplasm. The nucleus is typically flattened and located on the periphery. A typical fat cell is 0.1 mm in diameter with some being twice that size and others half that size. The fat stored is in a semi-liquid state and is composed primarily of triglycerides and cholesteryl ester. White fat cells secrete resistin, adiponectin, and leptin. An average adult has 30 billion fat cells with a weight of 30 lbs or 13.5 kg. If excess weight is gained as an adult, fat cells increase in size about fourfold before dividing and increasing the absolute number of fat cells present.

“Brown fat cells” or plurivacuolar or multilocular fat cells are typically polygonal in shape. Unlike white fat cells, these cells have considerable cytoplasm, with lipid droplets scattered throughout. The nucleus is round, and, although eccentrically located, it is not in the periphery of the cell. The brown color comes from the large quantity of mitochondria. Brown fat is used by an organism to generate heat and, in humans, is especially prominent in newborns and infants.

P2X purinoceptor 5 (“P2RX5 or P2X5”) belongs to the family of purinoceptors for ATP and functions as a ligand-gated ion channel. As referred to herein P2X5 refers to P2X5 as described in Le et al., FEBS Lett. 418 (1-2) 195-199 (1997) and its isoforms (NCBI reference sequences NP_{—}001191448.1; NP_{—}001191449.1; NP_{—}002552.2; NP_{—}778255.1)

The term “P2X5 targeting agent” refers to any molecule that binds P2X5 or variants there of with sufficient affinity and specificity. In certain embodiments, the P2X5 targeting agent is a peptide or polypeptide including but not limited to an antibody, antibody fragment, peptide mimetic and peptide analog. In another embodiment the P2X5 targeting agent is an aptamer.

Solute carrier family 36 (proton/amino acid symporter), member 2 (“Slc36a2” or “Slc36a2/PAT2”) is a pH-dependent proton-coupled amino acid transporter that belongs to the amino acid auxin permease 1 protein family. The encoded protein primarily transports small amino acids such as glycine, alanine and proline. Mutations in the gene encoding Slc36a2 are associated with iminoglycinuria and hyperglycinuria (Broer, Physiol Rev. 88(1):249-86 (2008)). Slc36a2 is 483 amino acids in length. The sequence is shown below (SEQ ID NO. 1) and is described in Boll et al. J. Biol. Chem. 277 (25), 22966-22973 (2002) and NCBI reference sequence number NP_{—}861441.2.

```
    1 msvtkstegp qgavaikldl msppesakkl enkdstflde
        spsesaglkk tkgitvfqal
  61 ihlvkgnmgt gilglplavk nagilmgpls llvmgfiach
        cmhilvkcaq rfckrlnkpf
121 mdygdtvmhg leanpnawlq nhahwgrhiv sffliitqlg
        fccvyivfla dnlkqvveav
181 nsttnncysn etviltptmd srlymlsflp flvllvlirn
        lriltifsml anismlvslv
241 iiiqyitqei pdpsrlplva swktyplffg taifsfesig
        vvlplenkmk narhfpails
301 lgmsivtsly igmaalgylr fgddikasis lnlpncwlyq
        svkllyiagi lctyalqfyv
361 paeiiipfai srvstrwalp ldlsirlvmv cltcllaili
        prldlvislv gsysgtalal
421 iippllevtt fysegmsplt ifkdalisil gfvgfvvgty
        qaldellkse dshpfsnstt
481 fvr
```

The term “Slc36a2 targeting agent” refers to any molecule that binds Slc36a2 or variants there of with sufficient affinity and specificity. In certain embodiments, the Slc36a2 targeting agent is a peptide or polypeptide including but not limited to an antibody, antibody fragment, peptide mimetic and peptide analog. In another embodiment the Slc36a2 targeting agent is an aptamer.

Solute carrier family 7 (neutral amino acid transporter light chain, asc system), member 10 (“Slc7a10 or Slc7a19/Asc1”) mediates high-affinity transport of D-serine and several other neutral amino acids (Nakauchi et al., Neurosci. Lett 287: 231-235 (2000) [PubMed 10863037]). Slc7a10 is 523 amino acids in length. The sequence is shown below (SEQ ID NO. 2) and is described in Fukasawa et al., J. Biol. Chem. 275 (13), 9690-9698 (2000) and NCBI reference sequence number NP_{—}062823.1.

```
    1 maghtqqpsg rgnprpapsp spvpgtvpga servalkkei
        gllsactiii gniigsgifi
  61 spkgvlehsg svglalfvwv lgggvtalgs lcyaelgvai
        pksggdyayv teifgglagf
121 lllwsavlim yptslavism tfsnyvlqpv fpncipptta
        srvlsmaclm lltwvnsssv
181 rwatriqdmf tggkllalsl iigvgllqif qghfeelrps
        nafafwmtps vghlalaflq
241 gsfafsgwnf lnyvteemvd arknlpraif isiplvtfvy
        tftniayfta mspqellssn
301 avavtfgekl lgyfswvmpv svalstfggi ngylftysrl
        cfsgareghl psllamihvr
361 hctpipallv ccgataviml vgdtytliny vsfinylcyg
        vtilgllllr wrrpalhrpi
421 kvnllipvay lvfwafllvf sfisepmvcg vgviiiltgv
        pifflgvfwr skpkcvhrlt
481 esmthwggel cfvvypqdap eeeengpcpp sllpatdkps
        kpq
```

The term “Slc7a10 targeting agent” refers to any molecule that binds Slc7a10 with sufficient affinity and specificity. In certain embodiments, the Slc7a10 targeting agent is a peptide or polypeptide including but not limited to an antibody, antibody fragment, peptide mimetic and peptide analog. In another embodiment the Slc7a10 targeting agent is an aptamer.

A “BAT agent” refers to any agent that modifies BAT development. A BAT agent can be an agent that increases or decreases brown adipose tissue (BAT) mass and or BAT activity. In one embodiment, the BAT agent is an agent that increases BAT mass. In another embodiment that BAT agent is an agent that promotes BAT activity. In yet another embodiment, the BAT agent is an agent that both increases BAT mass and increases BAT activity. Examples of BAT agents include but are not limited to agents that 1) increase the level of BAT differentiation in a cell or cell population; 2) increase BAT mass; and or 3) enhance the thermogenic potential (activity) of existing BAT tissue. BAT mass and activity can be measured by any of a number of techniques known in the art, including measuring any of, e.g. UCP-1 expression, BAT morphology or BAT thermodynamics, e.g. cytochrome oxidase activity, Na+K+ATPase enzyme units, or other enzymes involved in BAT thermogenesis, or in vivo by PET/CT scanning or thermography.

In certain embodiments, the BAT agent is a bone morphogenic protein (“BMP”), including but not limited to BMP-2, BMP-4, BMP-5, BMP-6 and BMP-7. More specifically including human mature BMP-2, BMP-4, BMP-5, BMP-6 and BMP-7. BMP proteins have been used in the clinic in the treatment of bone and cartilage disorders or wounds. The effective clinical use of recombinant BMPs is discussed in Einhorn, J. Bone and Joint Surgery 85A:82-88 (2003), and Sandhu, Spine 28 (15):S64-73 (2003). More recently, BMPs has been discovered to play an important role in promoting brown adipocyte differentiation and thermogenesis in vivo. Tseng et al. Nature 454 (7202): 100-1004 (2008). Furthermore, Tseng et al. demonstrated that adenoviral-mediated expression of BMP-7 in mice resulted in significant increases in brown, but not white fat mass, and lead to increased energy expenditure and reduced weight gain. A BMP polypeptide (e.g., a mature BMP polypeptide) is itself is a viable therapeutic agent because BMPs are small secreted proteins that are internalized into their target cells where they exert their activity.

“BMP-2” is 396 amino acids in length, localized to chromosome 20p12 in human. The nucleotide and amino acid sequences of human BMP-2 are disclosed in Wozney et al., Science 242 (4885):1528-1534 (1988). BMP2 belongs to the transforming growth factor-beta (TGFB) superfamily. Bone morphogenetic protein induces bone formation, and BMP2 is a candidate gene for the autosomal dominant disease of fibrodysplasia (myositis) ossificans progressive. Bone morphogenetic protein 2 regulates myogenesis through dosage-dependent PAX3 expression in pre-myogenic cells, and is expressed in mesoderm under SHM control through the SOX9.

The human BMP-2 is shown below (SEQ ID NO. 3). Amino acids 38-268 are the TGF-beta propeptide domain, and 291-396 are the TGF-beta family N-terminal domain. Amino acids 283-396 are the mature peptide. The sequence is set forth in Wozney et al., Science 242:1528-1534 (1988) and NBCI Sequence No. NP_{—}001191.1.

```
    1 mvagtrclla lllpqvllgg aaglvpelgr rkfaaassgr
        pssqpsdevl sefelrllsm
  61 fglkqrptps rdavvppyml dlyrrhsgqp gspapdhrle
        raasrantvr sfhheeslee
121 lpetsgkttr rfffnlssip teefitsael qvfreqmqda
        lgnnssfhhr iniyeiikpa
181 tanskfpvtr lldtrlvnqn asrwesfdvt pavmrwtaqg
        hanhgfvvev ahleekqgvs
241 krhvrisrsl hqdehswsqi rpllvtfghd gkghplhkre
        krqakhkqrk rlkssckrhp
301 lyvdfsdvgw ndwivappgy hafychgecp fpladhlnst
        nhaivqtivn svnskipkac
361 cvptelsais mlyldenekv vlknyqdmvv egcgcr
```

“BMP-4” induces cartilage and bone formation, and is important in mesoderm induction, tooth development, limb formation and fracture repair. The sequence of the BMP-4 preproprotein is shown below (SEQ ID. NO. 4). Amino acids 41-276 are the TGF-beta propeptide domain, and 302-408 are the TGF-beta family N-terminal domain. Amino acids 293-408 are the mature peptide. The sequence is set forth in Wozney et al., Science 242:1528-1534 (1988) and NCBI Sequence No. NP_{—}001193.2

```
    1 mipgnrmlmv vllcqvllgg ashaslipet gkkkvaeiqg
        haggrrsgqs hellrdfeat
  61 llqmfglrrr pqpsksavip dymrdlyrlq sgeeeeeqih
        stgleyperp asrantvrsf
121 hheehlenip gtsensafrf lfnlssipen evissaelrl
        freqvdqgpd wergfhrini
181 yevmkppaev vpghlitrll dtrlvhhnvt rwetfdvspa
        vlrwtrekqp nyglaievth
241 lhqtrthqgq hvrisrslpq gsgnwaqlrp llvtfghdgr
        ghaltrrrra krspkhhsqr
301 arkknkncrr hslyvdfsdv gwndwivapp gyqafychgd
        cpfpladhln stnhaivqtl
361 vnsvnssipk accvptelsa ismlyldeyd kvvlknyqem
        vvegcgcr
```

The “BMP-5” preproprotein is a 454 amino acid protein, as shown below (SEQ ID. NO. 5). BMP-5 induces cartilage and bone formation. The sequence is set forth in Celeste et al., Proc. Natl. Acad. Sci. U.S.A., 87, 9843-9847 (1990) and NCBI Sequence No. NP_{—}066551.1. The mature BMP-5 protein is believed to be amino acids 323-454 of SEQ ID NO: 5, and has four potential N-linked glycosylation sites per polypeptide chain, and four potential disulfide bridges. See UniProt Accession Nos. P22003; Q9H547; or Q9NTM5.

```
    1 mhltvfllkg ivgflwscwv lvgyakgglg dnhvhssfiy
        rrlrnherre igreilsilg
  61 lphrprpfsp gkqassap1f mldlynamtn eenpeeseys
        vraslaeetr garkgypasp
121 ngyprriqls rttplttqsp plaslhdtnf lndadmvmsf
        vnlverdkdf shqrrhykef
181 rfdltqiphg eavtaaefri ykdrsnnrfe netikisiyq
        iikeytnrda dlflldtrka
241 qaldvgw1vf ditvtsnhwv inpqnnlglq lcaetgdgrs
        invksaglvg rqgpqskqpf
301 mvaffkasev llrsvraank rknqnrnkss shqdssrmss
        vgdyntseqk qackkhelyv
361 sfrdlgwqdw iiapegyaaf ycdgecsfpl nahmnatnha
        ivqtlvhlmf pdhvpkpcca
421 ptklnaisvl yfddssnvil kkyrnmvvrs cgch
```

“BMP-6” is an autocrine stimulator of chondrocyte differentiation, and is involved in the development of embryonic neural, and urinary systems, as well as growth and differentiation of liver and keratinocytes. The sequence is set forth in Celeste et al., Proc. Natl. Acad. Sci. U.S.A., 87, 9843-9847 (1990) and NCBI Sequence No. NP_{—}001709.1. The human BMP-6 precursor is shown below (SEQ ID NO. 6). The mature polypeptide is believed to include amino acids 374-513 of SEQ ID NO: 6. Other active BMP-6 polypeptides include polypeptides including amino acids 382-513, 388-513 and 412-513 of SEQ ID NO:6. Administration, antisense treatment, and quantitation of BMP-6 are described in Boden et al. Endocrinology Vol. 138, No. 7 2820-2828.

```
    1 mpglgrraqw lcwwwgllcs ccgppplrpp lpaaaaaaag
        gqllgdggsp grteqpppsp
  61 qsssgflyrr lktqekremq keilsvlglp hrprplhglq
        qpqppalrqq eeqqqqqqlp
121 rgepppgrlk saplfmldly na1sadnded gasegerqqs
        wpheaasssq rrqpppgaah
181 plnrksllap gsgsggaspl tsaqdsafln dadmvmsfvn
        lveydkefsp rqrhhkefkf
241 nlsqipegev vtaaefriyk dcvmgsfknq tflisiyqvl
        qehqhrdsdl flldtrvvwa
301 seegwlefdi tatsnlwvvt pqhnmglqls vvtrdgvhvh
        praaglvgrd gpydkqpfmv
361 affkvsevhv rttrsassrr rqqsrnrstq sqdvarvssa
        sdynsselkt acrkhelyvs
421 fqdlgwqdwi iapkgyaany cdgecsfpln ahmnatnhai
        vqtlvhlmnp eyvpkpccap
481 tklnaisvly fddnsnvilk kyrnmvvrac gch
```

“BMP-7” also belongs to the TGF-beta superfamily. It induces cartilage and bone formation, and may be the osteoinductive factor responsible for the phenomenon of epithelial osteogenesis. BMP-7 plays a role in calcium regulation and bone homeostasis, and in the regulation of anti-inflammatory response in the adult gut tissue. More recently it has been shown to play an important role in adipocyte differentiation. In particular, it has been found to promote brown adipocyte differentiation and thermogenesis in vivo and in vitro and inhibits white adipose tissue (WAT) adipogenesis (See Tseng et al. Nature 454 (7207): 1000-1004 (2008) and U.S. Pat. No. 7,576,052). The sequence of BMP-7 is shown below (SEQ ID NO: 7) and is described in Ozkaynak et al. EMBO J., 9(&): 2085-2093 (1990) and NCBI Sequence No. NP_{—}001710.1. Amino acids 1-29 are a potential signal sequence; 30-431 are the prepropeptide, and 293-431 are the mature protein. The mature form of BMP-7 contains four potential N-linked glycosylation sites per polypeptide chain, and four potential disulfide bridges. See UniProt Accession No. P18075.

```
    1 mhvrslraaa phsfvalwap lfllrsalad fsldnevhss
        fihrrlrsqe rremqreils
  61 ilglphrprp hlqgkhnsap mfmldlynam aveegggpgg
        qgfsypykav fstqgpplas
121 lqdshfltda dmvmsfvnlv ehdkeffhpr yhhrefrfdl
        skipegeavt aaefriykdy
181 irerfdnetf risvyqvlqe hlgresdlfl ldsrtlwase
        egwlvfdita tsnhwvvnpr
241 hnlglqlsve tldgqsinpk lagligrhgp qnkqpfmvaf
        fkatevhfrs irstgskqrs
301 qnrsktpknq ealrmanvae nsssdqrqac kkhelyvsfr
        dlgwqdwiia pegyaayyce
361 gecafplnsy mnatnhaivq tlvhfinpet vpkpccaptq
        lnaisvlyfd dssnvilkky
421 rnmvvracgc h
```

In another embodiment, the BAT agent can be a “fibroblast growth factor”, including, but not limited to, FGF-6, FGF-9.

FGF-6 is a member of the fibroblast growth factor (FGF) family. FGF family members possess broad mitogenic and cell survival activities, and are involved in a variety of biological processes, including embryonic development, cell growth, morphogenesis, tissue repair, tumor growth and invasion. This gene displayed oncogenic transforming activity when transfected into mammalian cells. The amino acid sequence is shown below (SEQ ID NO: 8).

```
    1 malgqklfit msrgagrlqg tlwalvflgi lvgmvvpspa
        gtranntlld srgwgtllsr
  61 sraglageia gvnwesgylv gikrqrrlyc nvgigfhlqv
        lpdgrisgth eenpysllei
121 stvergvvsl fgvrsalfva mnskgrlyat psfqeeckfr
        etllpnnyna yesdlyqgty
181 ialskygrvk rgskvspimt vthflpri
```

“FGF-9” is a 208 amino acid protein. The amino acid sequence is shown below (SEQ ID NO: 9).

```
    1 maplgevgny fgvqdavpfg nvpvlpvdsp vllsdhlgqs
        eagglprgpa vtdldhlkgi
  61 lrrrqlycrt gfhleifpng tiqgtrkdhs rfgilefisi
        avglvsirgv dsglylgmne
121 kgelygsekl tqecvfreqf eenwyntyss nlykhvdtgr
        ryyvalnkdg tpregtrtkr
181 hqkfthflpr pvdpdkvpel ykdilsqs
```

In another embodiment, the BAT agent can a small molecule ligand, that for example, increases BAT mass and or BAT activity including, for example, increasing the level of BAT differentiation in a cell or cell population to increase mass or enhance the thermogenic potential (activity) of existing BAT tissue. In a non-limiting example, the small molecule ligand can be, for example, a thiazolidinedione.

In another embodiment, the BAT agent can be a small interfering RNA (siRNA) that targets a gene that modifies brown adipose or brown adipocyte development. In a particular embodiment the siRNA targets a gene that increases or decreases BAT mass and or increases or decreases BAT activity including, for example, increasing the level of BAT differentiation in a cell or cell population to increase mass or enhance the thermogenic potential (activity) of existing BAT tissue.

A “WAT agent” refers to an agent that modifies white adipose or white adipocyte development. A WAT agent can, for example, either increase or decrease lipid storage and or increase or decrease adipokine secretion. In certain embodiments, the WAT agent is a growth factor and or hormone that modifies white adipose or white adipocyte development. In a particular embodiment the WAT agent can either increase or decrease lipid storage and or increase or decrease adipokine secretion, including for example insulin, BMPs and FGFs. In another embodiment the WAT agent is a small molecule ligand that can either increase or decrease lipid storage and or increase or decrease adipokine secretion (e.g. thiazolidinediones). In yet another embodiment the WAT agent is a small interfering RNA that targets a gene that modifies white adipose or white adipocyte development (e.g. resistin, RBP4, TNFalpha, IL-6).

### II. MODES FOR CARRYING OUT THE INVENTION

A. Identification of Novel Brown and White Adipocyte Specific Cell Surface Markers

Using a combination of in silico, in vitro and in vivo techniques, we have identified novel brown and white adipocyte specific cell surface markers. Specifically we identified that the ligand-gated ion channel P2X5 and the small amino acid transporter Slc36a2 are highly expressed in brown adipocytes and to a much lesser extent in other tissues and cell types including white adipocytes. In addition we identified that the small amino acid transporter Slc7a10 is highly expressed in white adipocytes and to a much lesser extent in other tissues and cell types including brown adipocytes.

B. Screening for Targeting Agents

In a particular embodiment, the present invention contemplates methods for screening for targeting agents that recognize, bind to or otherwise interact with the white and brown cell surface markers identified herein and specifically including newly discovered BAT surface markers P2X5 (a “P2X5 targeting agent”) and Slc36a2 (a “Slc36a2 targeting agent”) and WAT surface marker Slc7a10 (a “Slc7a10 targeting agent”). The screened and identified targeting agents may be selected from, for example, antibodies, antibody fragment, small peptides, small molecules (organic and non-organic), oligonucleotides, aptamers selected via screens of small combinatorial libraries and fusion proteins.

In another embodiment of the invention the targeting agent may be an agent that influences physiological function of the surface markers, P2X5, Slc36a2 and Slc7a10, by inhibiting or augmenting (partially or fully) downstream activities of the identified surface markers. That is, for example, by influencing signaling pathways that are activated, increased, decreased or inhibited by the identified surface markers. The agents may or may not work by entering the cell and directly or indirectly interacting with constituents of the downstream pathway(s). Agents that work without entering the cell may work by inducing or inhibiting (fully or partly) other competing pathways via interaction with other cell surface molecules or with agents that interact with other cell surface molecules. Agents that work by entering the cell may work by interacting with downstream elements or molecules that interact with downstream elements. Additionally, agents that affect transcription and/or translation of the identified cell surface markers, competing markers or pathway constituents may also influence the activities of the identified cell surface markers. It is noted here that the phrase “competes with” or similar, may mean working in opposition to the identified marker and associated pathway to partly or fully inhibit the activity; likewise, the phrase may mean working in conjunction with the identified marker and associated pathway to partly or fully augment the activity. Both meanings are assumed unless noted differently herein by the context in which the phrase is used.

Exemplary Screening Methods

Yeast Two-Hybrid System

A Yeast two-hybrid system (commercially available from Clontech) allows for the detection of protein-protein interactions in yeast. See generally, Ausubel, et al., Current Protocols in Molecular Biology (John Wiley & Sons) (pp. 13.14.1-13.14.14). The system can be used to screen specially constructed cDNA libraries for proteins that interact with a target protein (e.g., P2X5, Slc36a2 or Slc7a10 proteins or fragments thereof). The present invention contemplates the use of the two-hybrid system to screen for compounds that will bind to either the P2X5 protein, Slc36a2 protein or Slc7a10 protein. Compounds (e.g., proteins) identified in a two-hybrid screen which bind to the P2X5 protein, Slc36a2 protein or Slc7a10 protein may represent compounds capable of blocking or augmenting the signaling of P2X5 protein, Slc36a2 or Slc7a10 protein.

In Vitro Screening Methods

Protein interactions may also be detected using, for example, gel electrophoresis where protein interactions can be detected by changes in electrophoretic mobility (detecting, for example, changes in size due to the binding of one or more proteins with another protein). Additionally, protein interactions can be detected by Western blotting. Western blotting detects proteins by transferring proteins from an electrophoresis gel to, e.g., nitrocellulose paper. Antibodies are then used to detect proteins that may have been transferred to the paper. Co-localization of two or more antibodies indicates possible protein-protein interaction.

Similarly, protein-protein interactions can be detected using affinity column chromatography. In this procedure, a binding agent (e.g., a target protein such as P2X5, Slc36a2 or Slc7a10) is bound to the column media (usually, for example, sepharose beads treated to bind the selected target protein and then treated to block any unused binding sites). The protein (or a mixture of proteins) suspected of interacting with the selected target protein is then run over the column. Proteins capable of interacting with the selected protein will bind the target protein and non-interacting proteins will run through the column. Bound, interactive proteins can then be released by changing stringency conditions.

Combinatorial Peptide and Small Molecule Libraries

Another aspect of the present invention relates to identifying molecules which bind the markers identified herein by screening combinatorial polypeptide libraries which encode either a random or controlled collection of amino acids. One such method is identifying molecules which bind, for example, P2X5, Slc36a2 or Slc7a10 from a polypeptide array. An array of polypeptides is synthesized on a solid support (e.g., a biological chip) as described by Pirrung et al., U.S. Pat. No. 5,143,854 (1992), the contents of which are incorporated herein by reference. The polypeptides which are attached to the support are called the probe. The resulting product is then processed to determine which polypeptides of the array bind a target protein, in this case P2X5, Slc36a2 or Slc7a10. The array linked support is contacted with the target molecule under conditions appropriate for binding, and specific probe proteins which bind the target molecule are identified. Methods for detecting labeled markers on a support are provided by Trulson et al., U.S. Pat. No. 5,578,832 (1996), the contents of which are incorporated herein by reference.

Another method for identifying polypeptides from a library which bind to a specified molecule is provided by Dower et al., U.S. Pat. No. 5,432,018 (1995), the contents of which are incorporated herein by reference. In addition, libraries of non-polypeptide chemical compounds can be screened for binding to and/or inhibition of P2X5, Slc36a2 or Slc7a10 by the method of Zambias et al., U.S. Pat. No. 5,807,754 (1998), the contents of which are incorporated herein by reference, and also the method of J. Ellman, U.S. Pat. No. 5,288,514 (1994) the contents of which are incorporated herein by reference.

Targeting Brown Adipose Tissue and or Brown Adipocytes

In one embodiment, the invention contemplates using the newly identified brown adipose specific surface markers, P2X5 and P2X5 targeting agents, for therapeutic, diagnostic and screening purposes. In another embodiment, the invention contemplates using the newly identified brown adipose specific surface markers, Slc36a2 and Slc36a2 targeting agents, for therapeutic, diagnostic and screening purposes.

In a one embodiment, the invention contemplates administering to a subject a therapeutically effective amount of an agent to be targeted to a brown adipose tissue or brown adipocyte where the agent is coupled to a P2X5 targeting agent or a Slc36a2 targeting agents. The agent is preferably a BAT agent that modifies BAT activity and preferably a BAT agent that increases BAT mass, promotes BAT activity or both. Alternatively the BAT agent decreases BAT mass, inhibits BAT activity or both. In one aspect the BAT agent is a BMP, a growth factor, small molecule ligand or small siRNA. The P2X5 targeting agent and Slc36a2 targeting agent are preferably antibodies, antibody fragments or aptamers to P2X5 and Slc36a2 respectively.

In another embodiment, the invention contemplates using the brown adipose specific surface markers, P2X5 and Slc36a2, for targeting brown adipose tissue or brown adipocytes as a diagnostic tool. In one aspect the P2X5 and Slc36a2 targeting agents can be used in a diagnostic assay to evaluate the amount of brown adipose tissue in a subject. Varies art-recognized methods are available including for example radioactively or fluorescently labeling the P2X5 and Slc36a2 targeting agents (e.g. antibody, aptamer) and measuring the signal volume and intensity.

In another embodiment, the invention contemplates using the brown adipose specific surface markers, P2X5 and Slc36a2, for targeting brown adipose tissue or brown adipocytes in a subject as a screening tool. In one aspect P2X5 and Slc36a2 can be used to detect the presence or absence of brown adipose tissue in a subject.

In one embodiment the subject is a mammal. In another embodiment the subject is a human, a dog, a monkey, a mouse, a rat. In a further embodiment the agent is a BAT agent that binds a surface protein on BAT. In another embodiment the agent is a BAT agent that modifies brown adipose tissue or brown adipocyte development.

Targeting White Adipose Tissue and or White Adipocytes

In one embodiment, the invention contemplates using the newly identified white adipose specific surface marker, Slc7a10 and Slc7a10 targeting agents, for therapeutic, diagnostic and screening purposes.

In a one embodiment, the invention contemplates administering to a subject a therapeutically effective amount of an agent to be targeted to a white adipose tissue or white adipocyte where the agent is coupled to a Slc7a10 targeting agent. The agent is preferably a WAT agent that modifies WAT activity and preferably a WAT agent that increases or decreases lipid storage and or increases adipokine secretion. In one aspect the WAT agent is a growth factor, hormone, small molecule ligand, small siRNA or a toxin. The Slc7a10 targeting agent is preferably an antibody, antibody fragment or aptamer to Slc7a10.

In another embodiment, the invention contemplates using the white adipose specific surface marker, Slc7a10, for targeting brown adipose tissue or brown adipocytes as a diagnostic tool. In one aspect the Slc7a10 targeting agent can be used in a diagnostic assay to evaluate the amount of white adipose tissue in a subject. Varies art-recognized methods are available including for example radioactively or fluorescently labeling the Slc7a10 targeting agent (e.g. antibody, aptamer) and measuring the signal volume and intensity.

In another embodiment, the invention contemplates using the white adipose specific surface marker, Slc7a10, for targeting white adipose tissue or white adipocytes in a subject as a screening tool. In one aspect the Slc7a10 can be used to detect the presence or absence of white adipose tissue in a subject.

In one embodiment the subject is a mammal. In another embodiment the subject is a human, a dog, a monkey, a mouse, a rat.

Compositions that Modify BAT Development

The present invention contemplates a composition comprising a P2X5 targeting agent or a Slc36a2 targeting agent coupled to BAT agent. The invention further contemplates that the “BAT agents” include any molecule, including but not limited to small molecules, peptides, nucleotides, oligonucleotides, polypeptides and fusion proteins that modify BAT development such that the BAT development is either enhanced or inhibited. Methods for identifying agents that modify BAT development such that the BAT development is either enhanced or inhibited are known in the art including for example administering the agent of interest to a cell sample and measuring the UCP-1 expression, BAT morphology or BAT thermodynamics, e.g. cytochrome oxidase activity, Na+K+ATPase enzyme units.

Specifically contemplated are BAT agents that enhance BAT or brown adipocyte development by either increasing BAT or brown adipocyte mass or promoting BAT or brown adipocyte activity or both including but not limited to bone morphogenetic protein (BMP) polypeptides or fragment thereof, e.g., BMP-2, BMP-4, BMP-6, BMP-7; fibroblast growth factors, e.g., FGF-6, FGF-9; small molecule ligands, e.g., thiazolidinediones and siRNAs.

Specific examples of “BAT agents” that enhances BAT or brown adipocyte development include but are not limited to the following classes of molecules. (1) Bone morphogenic proteins (BMPs) including but not limited to (a) a BMP-2, -4, -6, and/or -7 polypeptide or a functional fragment or variant thereof, preferably an active (e.g., BMPR-I and/or BMPR-II activating) BMP-2, -4, -6, and/or -7 polypeptide or a functional fragment or analog thereof (e.g., a mature BMP-2, -4, -6, and/or -7 polypeptide, e.g., a mature BMP-2, -4, -6, and/or -7 polypeptide described herein); (b) a peptide or protein agonist of BMP-2, -4, -6, and/or -7 that increases the activity, e.g., the BMPR-I and/or BMPR-II activating activity of BMP-2, -4, -6, and/or -7 (e.g., by increasing or stabilizing binding of BMP-2, -4, -6, and/or -7 to its receptor); (c) a small molecule or protein mimetic that mimics BMP-2, -4, -6, and/or -7 signaling activity, e.g., BMPR-I and/or BMPR-II binding activity, or SMAD phosphorylating activity; (d) a small molecule that increases expression of BMP-2, -4, -6, and/or -7, e.g., by binding to the promoter region of a BMP-2, -4, -6, and/or -7 gene; (e) an antibody, e.g., an antibody that binds to and stabilizes or assists the binding of BMP-2, -4, -6, and/or -7 to a BMP-2, -4, -6, and/or -7 binding partner (e.g., a BMP-2, -4, -6, and/or -7 receptor described herein); or (f) a nucleotide sequence encoding a BMP-2, -4, -6, and/or -7 polypeptide or functional fragment or analog thereof. The nucleotide sequence can be a genomic sequence or a cDNA sequence. The nucleotide sequence can include: a BMP-2, -4, -6, and/or -7 coding region; a promoter sequence, e.g., a promoter sequence from a BMP-2, -4, -6, and/or -7 gene or from another gene; an enhancer sequence; untranslated regulatory sequences, e.g., a 5′ untranslated region (UTR), e.g., a 5′UTR from a BMP-2, -4, -6, and/or -7 gene or from another gene, a 3′ UTR, e.g., a 3′UTR from a BMP-2, -4, -6, and/or -7 gene or from another gene; a polyadenylation site; an insulator sequence. In another embodiment, the level of BMP-2, -4, -6, and/or -7 protein is increased by increasing the level of expression of an endogenous BMP-2, -4, -6, and/or -7 gene, e.g., by increasing transcription of the BMP-2, -4, -6, and/or -7 gene or increasing BMP-2, -4, -6, and/or -7 mRNA stability. In some embodiments, transcription of the BMP-2, -4, -6, and/or -7 gene is increased by: altering the regulatory sequence of the endogenous BMP-2, -4-6, and/or -7 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the BMP-2, -4, -6, and/or -7 gene to be transcribed more efficiently. In a preferred embodiment the agent is a BMP-2, -4, -6, and/or -7 polypeptide, preferably human, preferably a mature-2, -4, -6, and/or -7 polypeptide; a BMP-7 polypeptide that includes amino acids 293-431 of SEQ ID NO. 7. The polypeptide can be a recombinant polypeptide.

Compositions that Modify WAT Development

The present invention contemplates a composition comprising a Slc7a10 targeting agent coupled to WAT agent. The invention further contemplates that the “WAT agents” include any molecule, including but not limited to small molecules, peptides, nucleotides, oligonucleotides, polypeptides and fusion proteins that modify WAT development such that the WAT development is either enhanced or inhibited. Methods for identifying agents that modify WAT development such that the WAT development is either enhanced or inhibited are known in the art including for example administering the agent of interest to a cell sample and measuring the increase or decrease lipid storage or increased adipokine secretion.

Specifically contemplated are WAT agents that inhibit WAT or white adipocyte development by either decreasing WAT or white adipocyte mass or inhibiting WAT or white adipocyte activity or both. In a one embodiment the WAT agents include, but are not limited to, growth factors and hormones (e.g. insulin, BMPs, FGFs), small molecule ligands (e.g. thiazolidinediones), small interfering RNAs targeting for example resistin, RBP4, TNFalpha, IL-6) and toxins.

Administration

The agents described herein can be administered to a subject by standard methods. For example, the agent can be administered by any of a number of different routes including intravenous, intradermal, subcutaneous, percutaneous injection, oral (e.g., inhalation), transdermal (topical), and transmucosal. In one embodiment, the modulating agent can be administered orally. In another embodiment, the agent is administered by injection, e.g., intramuscularly, or intravenously. The agent can be encapsulated or injected, e.g., in a viscous form, for delivery to a chosen site, e.g., a site of adipose tissue, e.g., a subcutaneous or omentum adipose pad. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, cosmetic appearance and interface properties. One example is a collagen matrix.

The agent described herein can be incorporated into pharmaceutical compositions suitable for administration to a subject, e.g., a human. Such compositions typically include the polypeptide, nucleic acid molecule, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In some embodiments, the pharmaceutical composition is injected into a tissue, e.g., an adipose tissue.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, is relevant prior art for the present invention and the right to challenge the accuracy and pertinence of such patents, patent applications, publications, and other references is specifically reserved.

As used herein and in the appended claims, the singular forms “a”, “an”, and “the” include plural reference (and are equivalent to or have the same or similar meaning as the phrase “one or more”) unless the context clearly dictates otherwise.

The following examples should not be construed as limiting the scope of this disclosure.

### EXAMPLES

Methods

Real Time PCR

Seven week of C57/BL-6 mice were anesthetized and perfused with PBS. Tissues were collected and total RNA was extracted using the Qiagen RNeasy Mini kit. 1 ug of total RNA was reverse transcribed with random hexamere primers (Applied Biosystems) in 20 ul and subsequently diluted 1:10 with water. Real Time PCR was performed using 2×SYBR green (BioRad) and 2.5 ul of cDNA per well. Each sample was tested either in duplicates or triplicates. The following primers were used:

| | | |
|---|---|---|
| Tetra- | fwd | CCTTCCCAGAAAGGAAAAGG [SEQ ID NO: 10] |
| spanin | | |
| 18 | | |
| | rev | CAGGAGTCAACAGGATGCAA [SEQ ID NO: 11] |
| Frizzled | fwd | GACACGAGCTGCAGACAGAC [SEQ ID NO: 12] |
| Homolog 4 | | |
| | rev | TGGCACATAAACCGAACAAA [SEQ ID NO: 13] |
| P2X5 | fwd | CTGCAGCTCACCATCCTGT [SEQ ID NO: 14] |
| | rev | CACTCTGCAGGGAAGTGTCA [SEQ ID NO: 15] |
| CD300LG | fwd | GAGGCCTCTGGTCCTGCTAT [SEQ ID NO: 16] |
| | rev | ACGGTGTCACCTTCAAATCC [SEQ ID NO: 17] |
| Slc27a1 | Fwd | TTCTGCGTATCGTCTGCAAG [SEQ ID NO: 18] |
| | Rev | GCTCTAGCCGAACACGAATC [SEQ ID NO: 19] |
| ADRB3 | Fwd | TGAAACAGCAGACAGGGACA [SEQ ID NO: 20] |
| | Rev | GGCGTCCTGTCTTGACACTC [SEQ ID NO: 21] |
| AQP7 | Fwd | GCGAGAGTTTCTGGCAGAGT [SEQ ID NO: 22] |
| | Rev | CCGAGATAGCTGCCAGAGTT [SEQ ID NO: 23] |
| GPR119 | Fwd | GGACCGAAGCTGACATTTTG [SEQ ID NO: 24] |
| | Rev | TCCTCAGACCACTGGTAGCC [SEQ ID NO: 25] |
| Slc36a2 | Fwd | GTGCCAAGAAGCTGCAGAG [SEQ ID NO: 26] |
| | Rev | TGTTGCCTTTGACCAGATGA [SEQ ID NO: 27] |
| A53001 | Fwd | GCATGGTTGCTTCTTCCTTC [SEQ ID NO: 28] |
| 6l24rik | | |
| | Rev | GAGACTCGAGCTGCAGACCT [SEQ ID NO: 29] |
| NRG4 | Fwd | GCGGTCCTCCTCACTCTTAC [SEQ ID NO: 30] |
| | Rev | CAGGCTGATCTCACACTGGA [SEQ ID NO: 31] |
| GPR81 | Fwd | CCCTGAGCTTCACCTACCTG [SEQ ID NO: 32] |
| | Rev | TTCAGGCTGCAGATTGTGAG [SEQ ID NO: 33] |
| Slc7a10 | Fwd | GGGTGGCACTCAAGAAAGAG [SEQ ID NO: 34] |
| | Rev | AGTGTTCCAGGACACCCTTG [SEQ ID NO: 35] |
| Tmem120b | Fwd | CTGGAGGGAGAATTTCAGGA [SEQ ID NO: 36] |
| | Rev | GCTCCTCCAGCTTCTGCTTA [SEQ ID NO: 37] |
| hSlc7a10 | Fwd | GTGGCGCTCAAGAAGGAGAT [SEQ ID NO: 38] |
| | Rev | CCTTGGGCGAGATGAAGAT [SEQ ID NO: 39] |

Western Blotting

Seven week old C57/BL-6 mice were anesthetized and perfused with PBS. Tissues were collected and lysed in modified RIPA buffer containing 150 mM NaCl, 50 mM Tris pH 7.4, 1 mM EDTA, 1% Triton-X, 0.1% SDS and proteinase and phosphatase inhibitors (Sigma-Aldrich, St. Louis, Mo. 50 ug of protein lysate were loaded per well. PVDF membranes were blocked with 5% skim milk/PBS-T for one hour and incubated overnight with anti-Slc7a10 (1:1000, Abnova) or anti-P2X5 (1:1000, Abcam).

Adipocyte Isolation

Adipocytes from perigonadal, flank inguinal subcutaneous and brown adipose depots from each individual mouse were isolated by collagenase digestion (Geste, et al., Proc. Natl. Acad. Sci 103:6676-6681 (2006)).

Adipocyte Differentiation

3T3-L1 preadipocytes were cultured in DMEM 4.5 g glucose/liter and 10% FBS (growth medium). Twenty-four hours after reaching confluence differentiation was induced by addition of growth medium containing 100 nM Insulin, 500 μM IBMX (3-isobutyl-1-methylxanthine; a competitive, nonselective phosphodiesterase inhibitor) and 400 ng/ml Dexamethasone. The induction medium was replaced after 48 hours with growth medium containing 100 nM Insulin. Medium was changed every 48 hours.

### Example 1

### Identification of Adipocyte Specific Cell Surface Markers

The general approach used herein to identify white and brown adipocyte specific cell surface markers is based on a combination of in silico, in vitro and in vivo techniques. Initially, the Symatlas of gene expression was used and its successor the BioGPS (www.biogps.gnf.org) databases. These databases provide Affymetrix data from more than 100 different tissues and cell lines. In addition to normal searches these databases provide the possibility to search for genes with correlated gene expression profiles in comparison to a selected reference gene, such as UCP1 for brown fat and adiponectin for white fat.

White Adipocyte Tissue

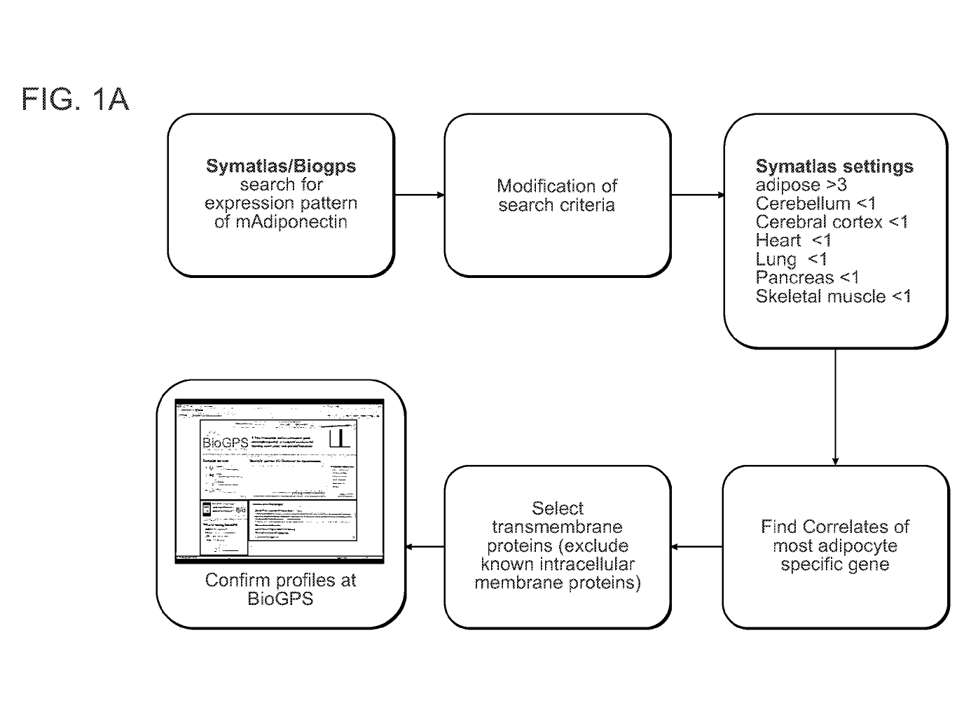

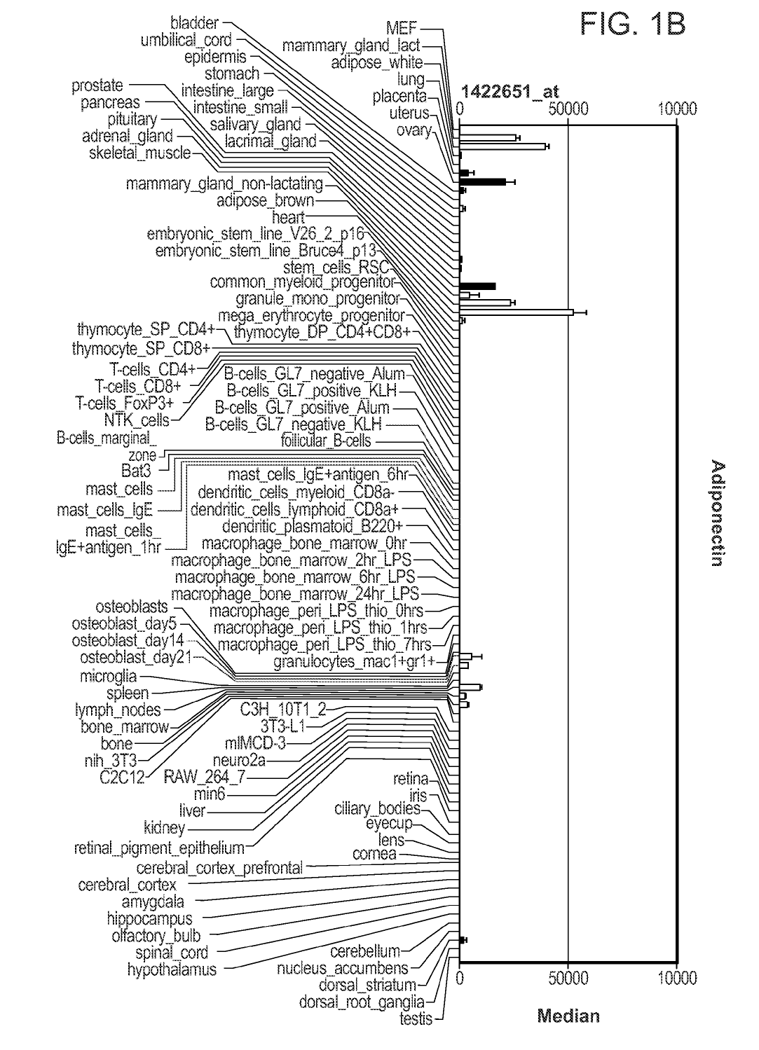

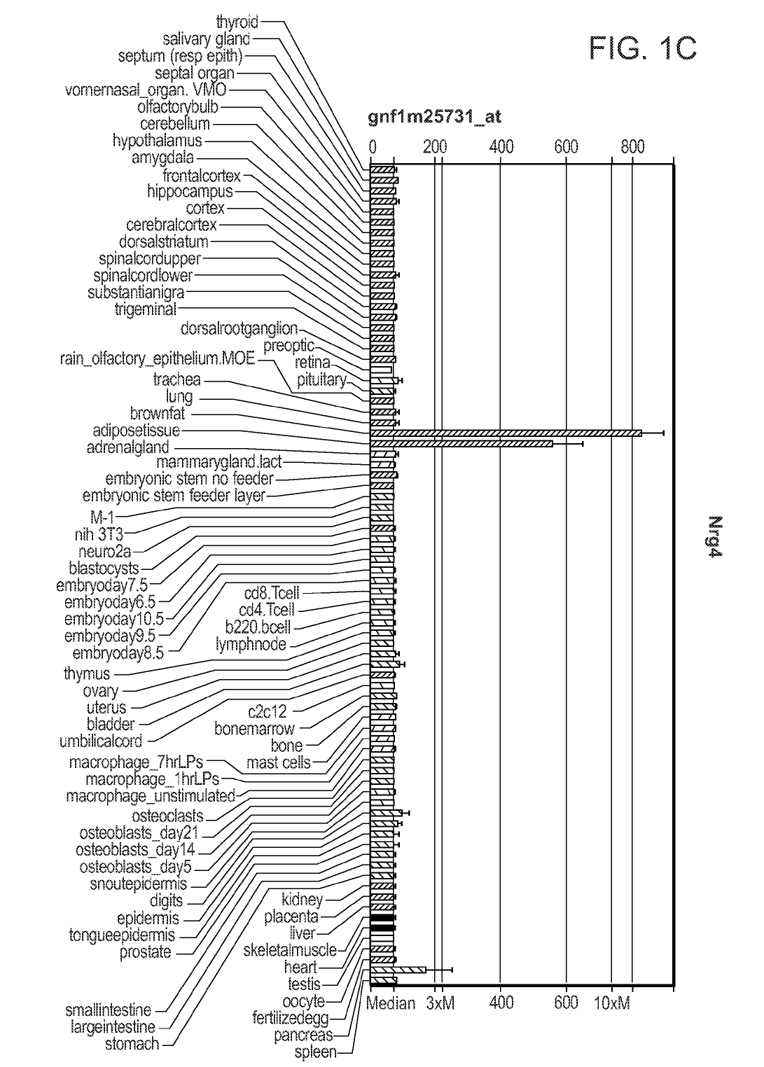

Initially genes were searched with a correlation coefficient greater 0.95 compared to adiponectin (as illustrated schematically in FIG. 1a). As illustrated in FIG. 1b, adiponectin expression was not restricted to adipose tissue; significant expression was also found in other tissues. Therefore, to achieve higher selectivity for adipose specific genes, searches were conducted for correlated genes with expression in adipose that was 3 times greater than the mean of all other tissues and cell lines tested, but that also had expression of less than the mean, i.e., <1, in cerebellum, cerebral cortex, heart, lung, pancreas and skeletal muscle. Using this gene set a subset of genes was identified that encoded cell surface, transmembrane proteins. Using this approach three transmembrane proteins were identified: [Nat8L (N-acetyltransferase 8-like), PRLR (prolactin receptor), Nrg4 (neuregulin 4)], from which Nrg4 showed highest adipose tissue specificity (FIG. 1c and other data not shown). To increase the number of potential adipocyte specific surface proteins, the search was repeated for genes that correlated genes with neuregulin 4. We identified seven candidate genes: Tmem120b (transmembrane protein 120B); ADRB3 (adrenergic beta-3-receptor); AQP7 (aquaporin 7); GPR81 (G protein-coupled receptor 81); SLC27a1/FATP1 (solute carrier family 27member 1/fatty acid transporter); Slc7a10 (solute carrier family 7member 10); neutral amino acid transporter, y+ system; A530016L24Rik.

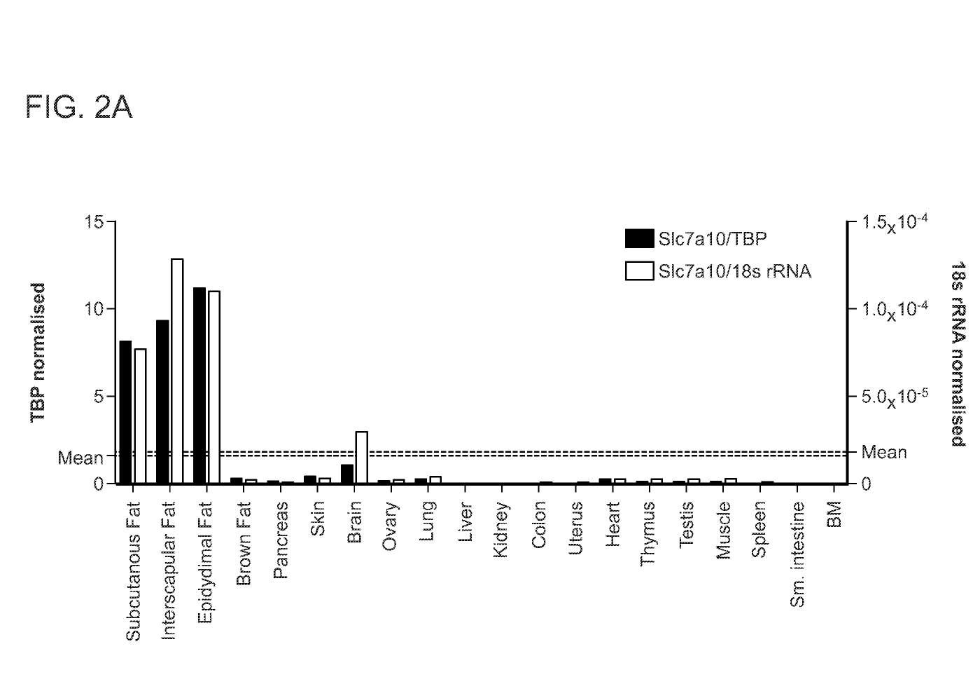

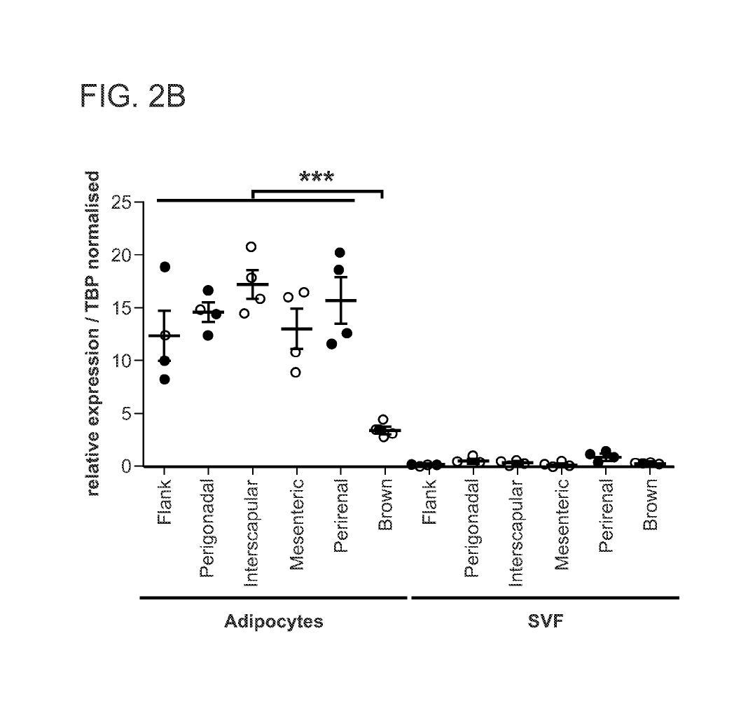

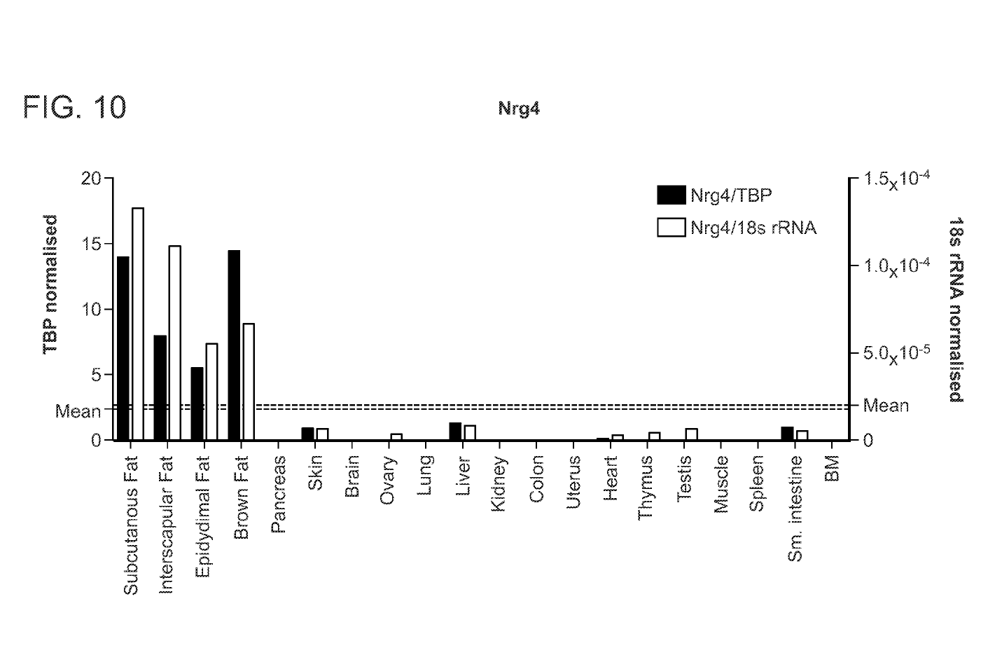

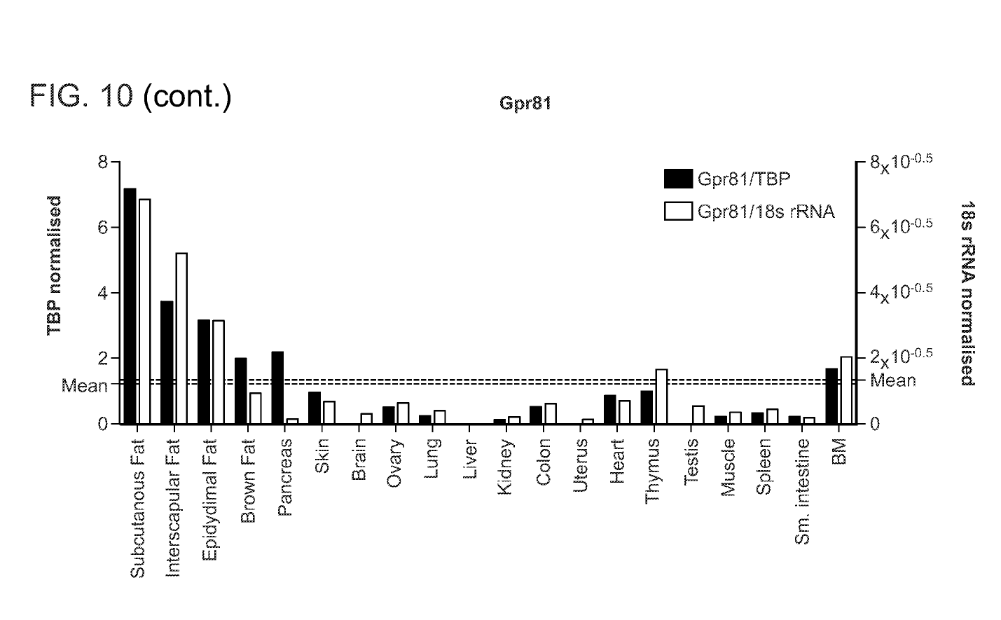

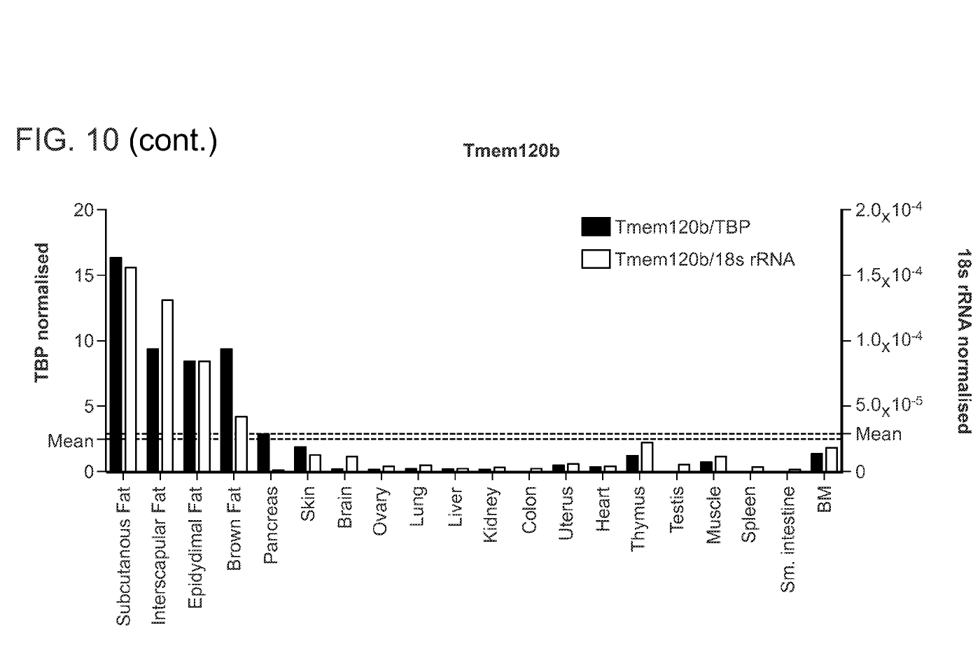

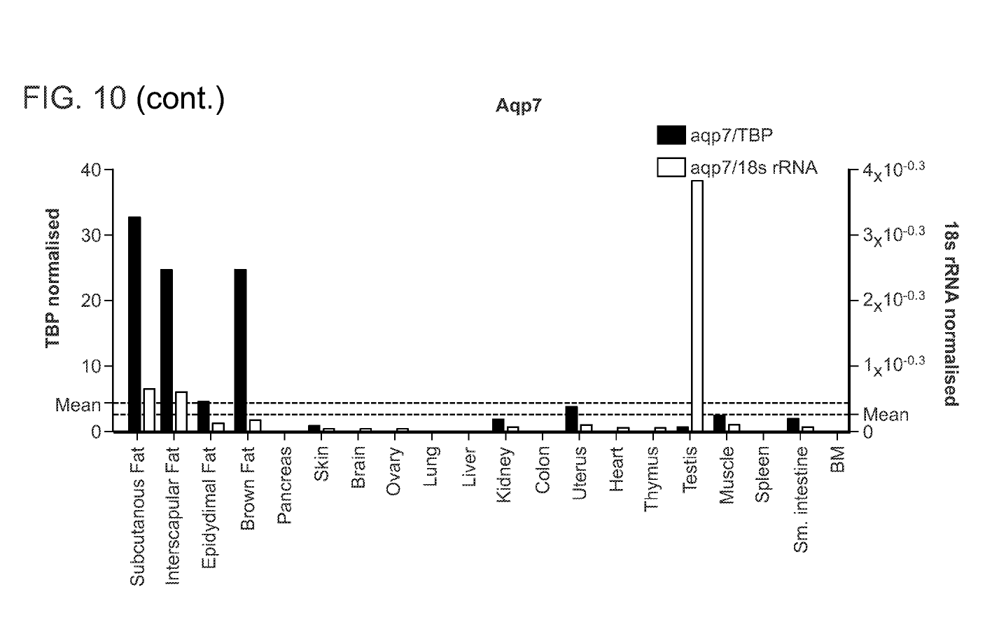

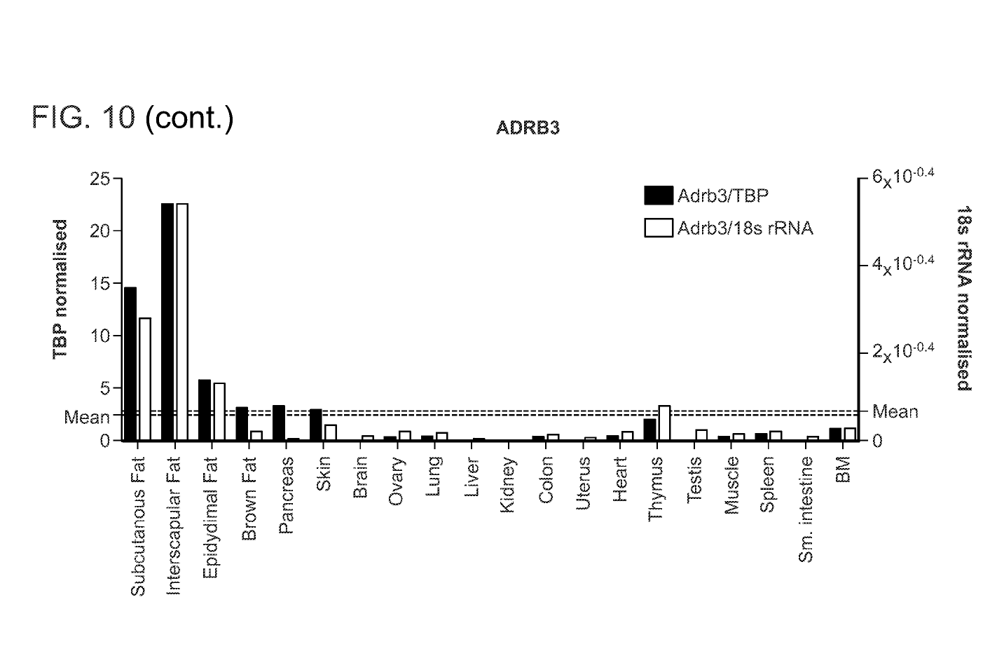

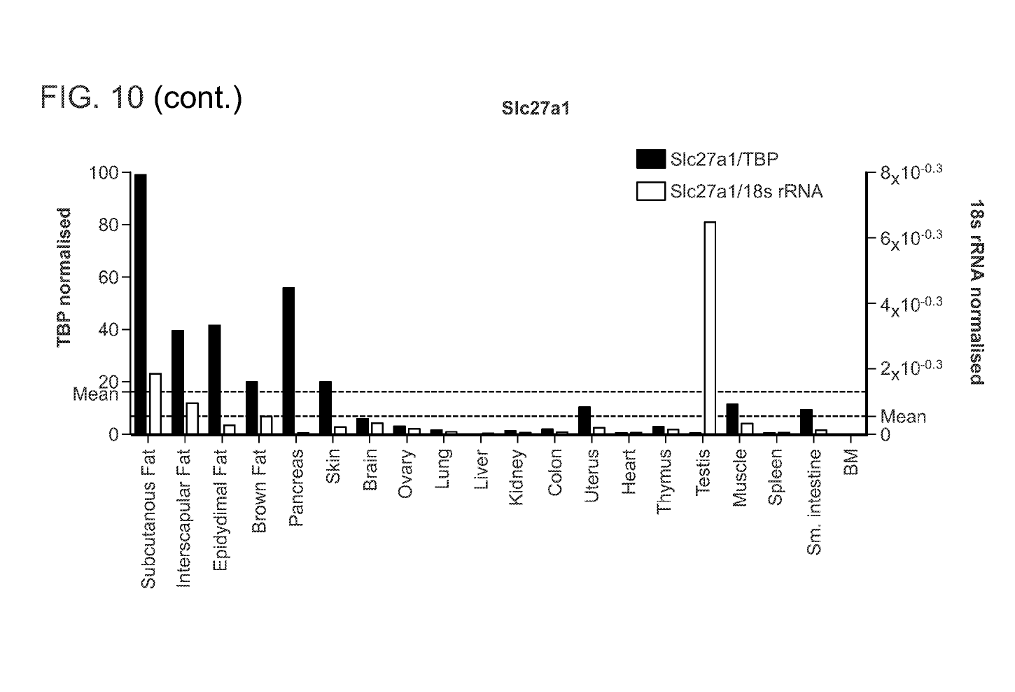

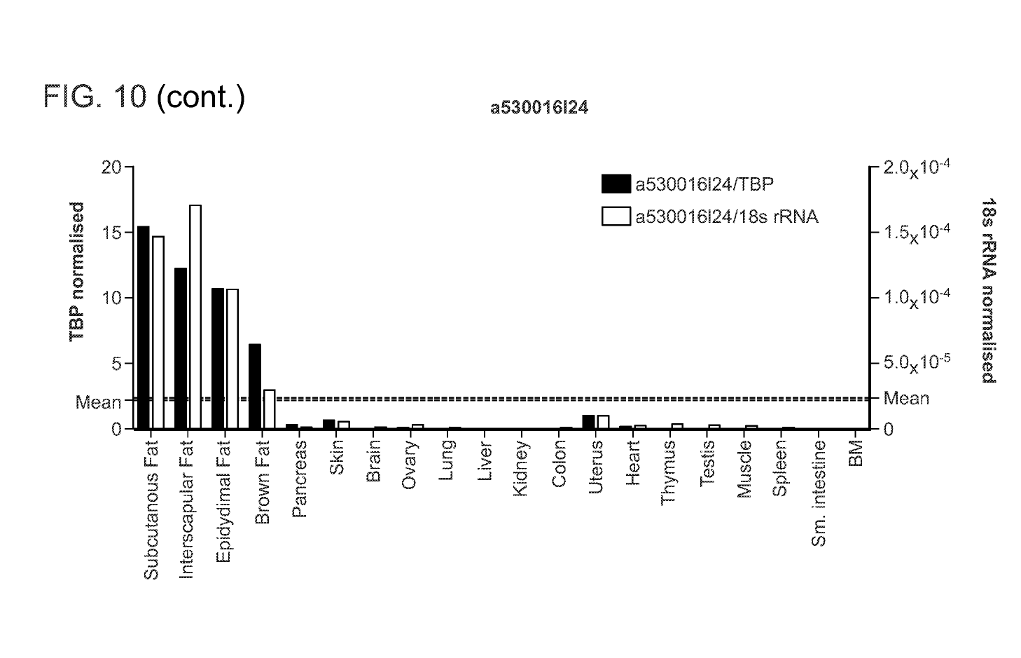

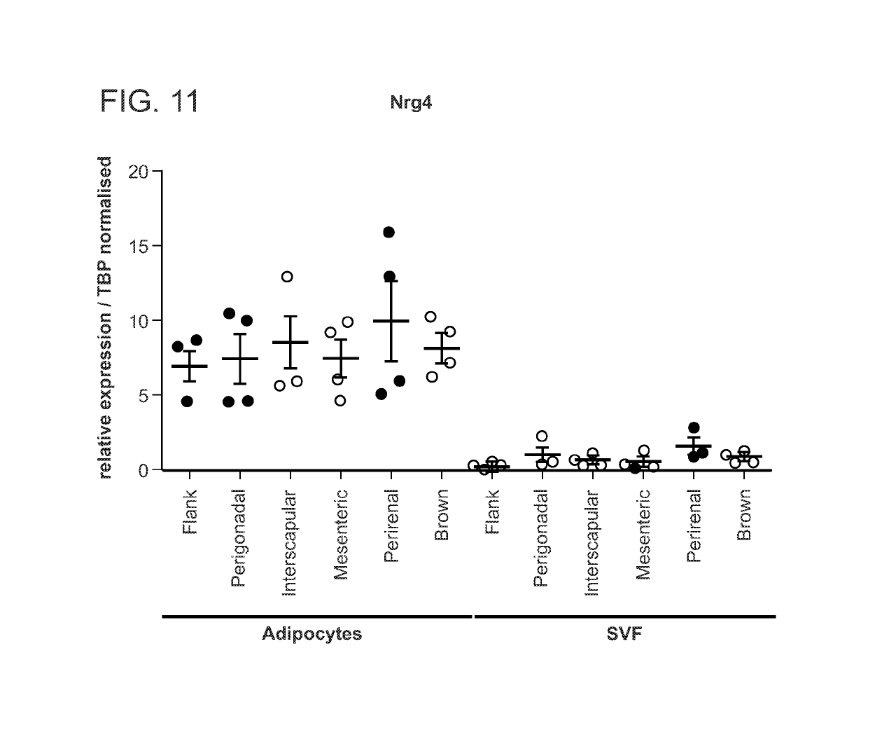

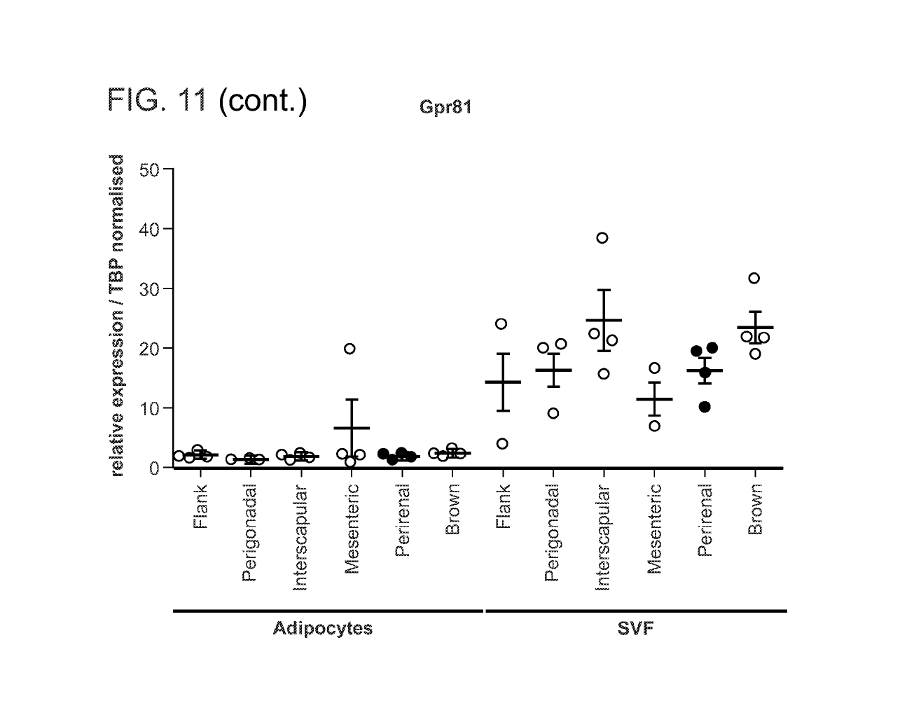

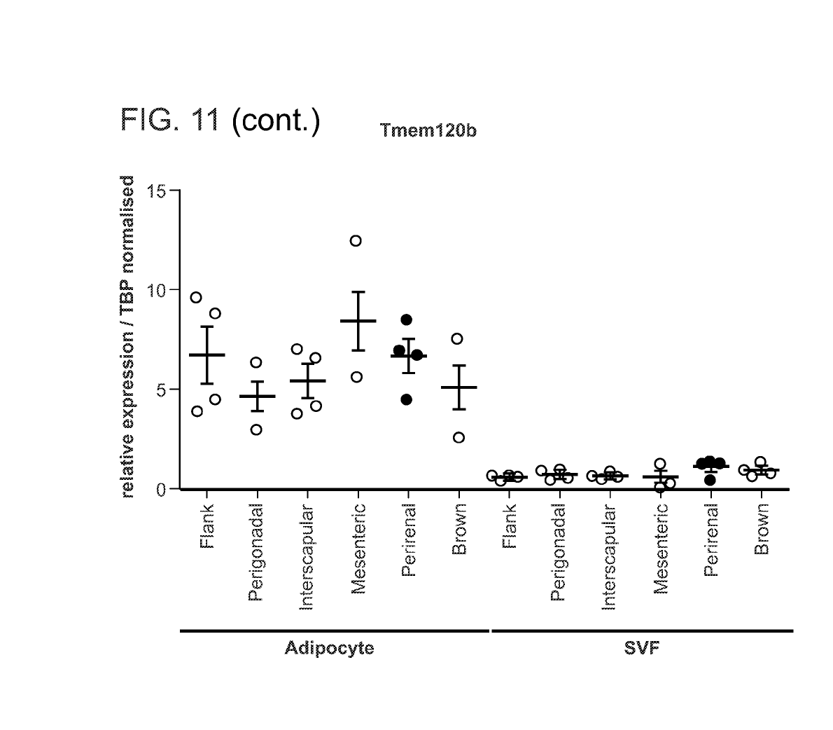

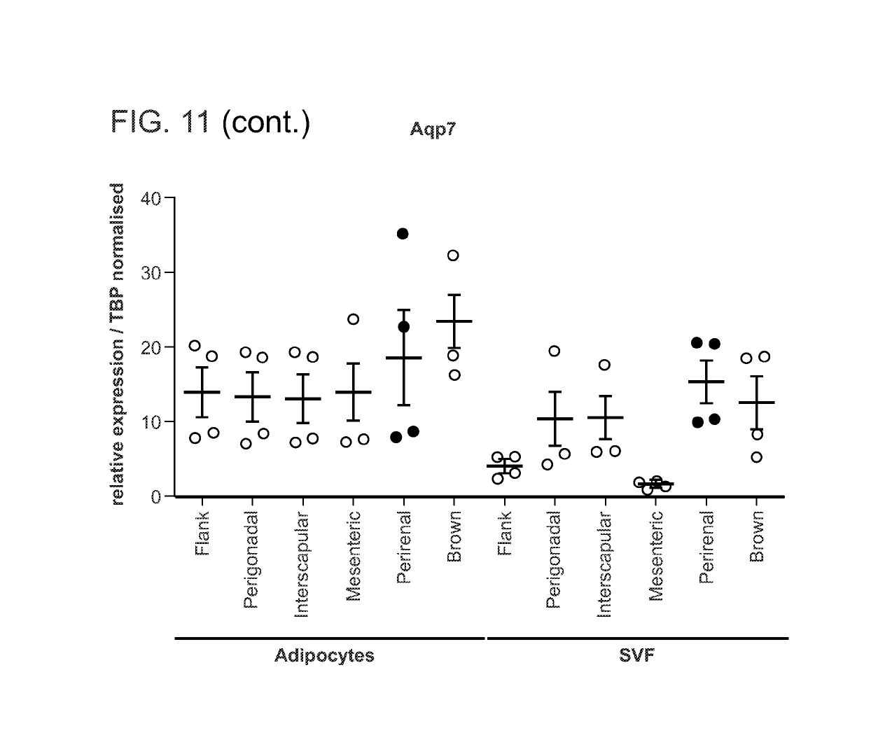

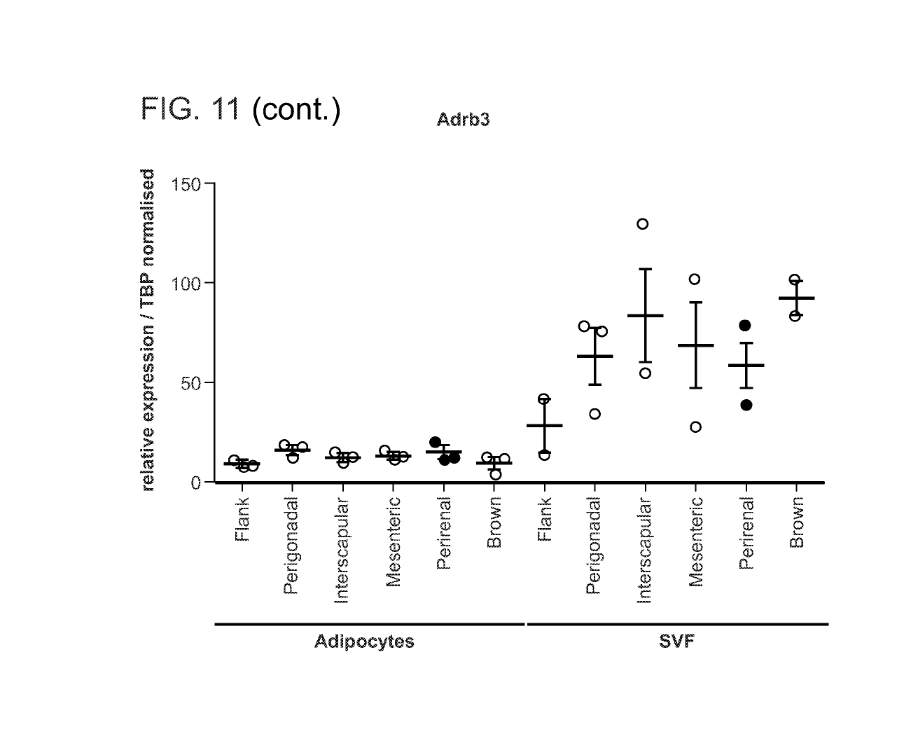

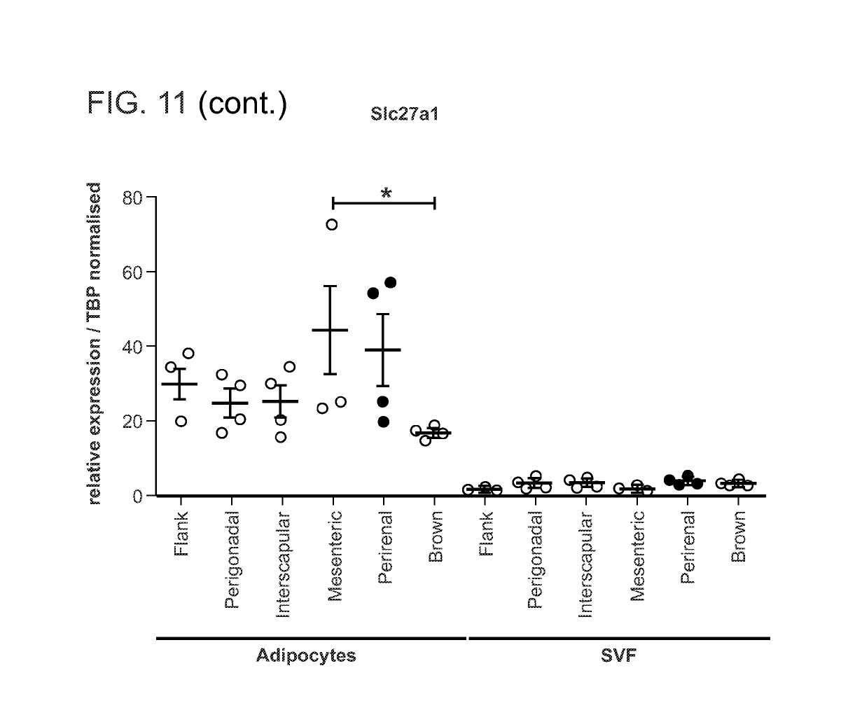

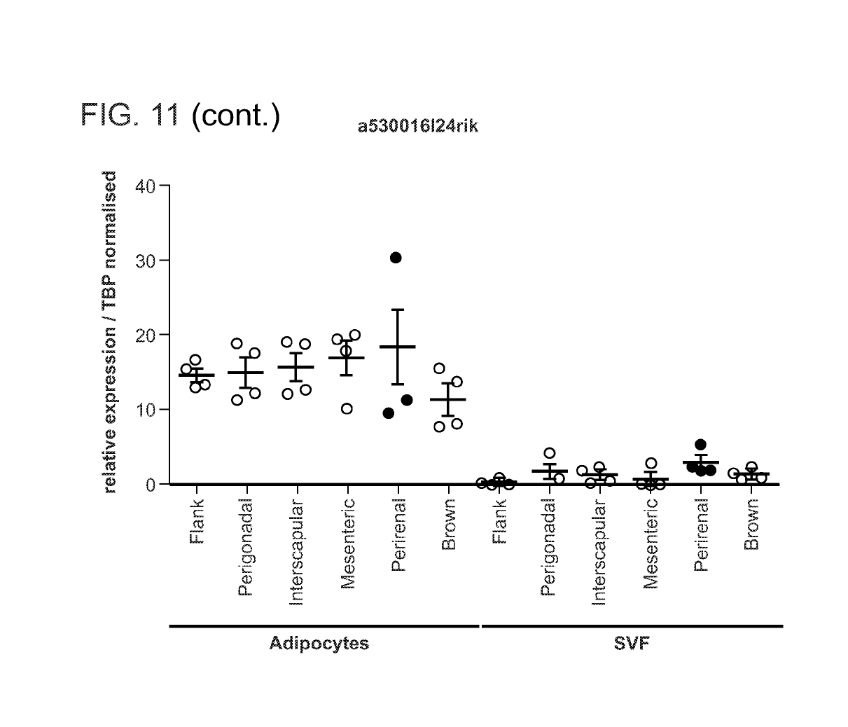

To confirm the Afffymetrix data, RNA was isolated from twenty tissues of seven week old C57/Bl6 male and female, PBS perfused mice. While all selected genes showed a higher expression in adipose tissue in comparison to other tissues tested, Nrg4, Aqp7, a530016124 and Slc7a10/Asc-1 showed greatest adipose tissue specificity (FIG. 2a and FIG. 10). Since adipose tissue is composed of adipocytes, as well as a variety of stromovascular cells (blood cells, immune cells, endothelial cells and preadipocytes), RNA was isolated from purified adipocytes and the stromal vascular fraction (SVF): in addition to any adipocytes the SVF may contain a variety of other cell type including pre-adipocytes, endothelial cells, smooth muscle cells, pericytes, fibroblasts and adult stem cells (ASCs). In addition, the SVF may also contain blood cells from the capillaries supplying the fat cells. These may include erythrocytes or red blood cells, B and T cells, macrophages, monocytes, mast cells, natural killer (NK) cells, hematopoietic stem cells and endothelial progenitor cells; to test for the adipocyte specificity of our candidate genes. As shown in FIG. 2b and FIG. 11, Gpr81, Adrb3 and Aqp7 had higher expression in the SVF than in the isolated adipocytes whereas expression of Nrg4, Slc27a1, a530016124 and Slc7a10 was specific to adipocytes. Nrg4, Slc27a1 and a530016124 resembled general adipocyte markers. In contrast Slc7a10 (FIG. 2b) was expressed at significantly higher levels in white adipocytes than in brown adipocytes and, thus, as a result of the work described herein, was identified for the first time as a white adipocyte surface marker.

Although the present inventors do not wish to be limited by theory, Slc7a10 is known in the art as Na⁺-independent neutral amino acid exchange transporter that forms a heterodimer with the 4F2hc protein. Slc7a10 has been shown to transport small neutral amino acids such as Gly, L-Ala, L-Ser, L-Thr, and L-Cys, and α-aminoisobutyric acid. In contrast to other members of the family of amino acid transporters associated with type II membrane glycoproteins (LAT family), Slc7a10 shows the highest affinity for D-serine, an activator of glutamate/N-methyl-D-aspartate (NMDA) receptors in the brain (Fukasawa, et al., J. Biol. Chem 275:9690-9698 (2000); Nakauchi, et al., Neurosci. Lett. 287: 231-235 (2000); Rutter, et al., Eur J Neurosci. 25:1757-1766 (2007)). In line with this, mice deficient for Slc7a10 (i.e., knockout mice) showed tremors, seizures and reduced body weight leading to lethality within 30 days after birth (Xie, et al., Brain Res 1052:212-221 (2005)). Expression analysis in mice and men revealed expression of Slc7a10 in brain, lung, small intestine, placenta, kidney and heart. Notably, however, the tissue panels used for these Northern Blot experiments did not include adipose tissue ((Fukasawa, et al., J. Biol. Chem 275:9690-9698 (2000); Nakauchi, et al., Neurosci. Lett. 287: 231-235 (2000). In the present application, Real-Time PCR analysis of Slc7a10 expression revealed high expression in adipose tissue, as well as additional expression in skin, brain, ovary, lung, heart, thymus, testis and skeletal muscle. As illustrated in FIG. 2a, expression levels in these tissues were around five times lower than in brain and around 30 times lower than in white adipose tissue.

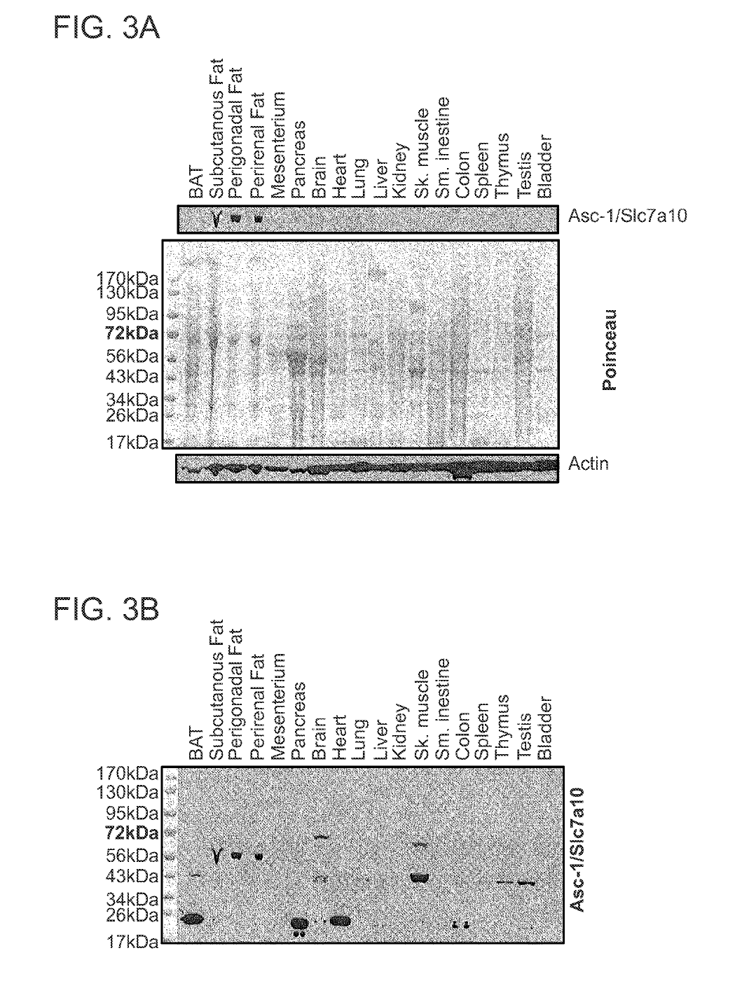

Next it was tested if the tissue distribution of Slc7a10 protein corresponds to the observed mRNA expression pattern. A panel of 18 tissues of perfused C57/BL-6 male and female mice was used. The predicted molecular weight of Slc7a10 is between 56-63 kDa. As shown in FIG. 3a strong signal was observed around ˜60 kDa in subcutaneous, perigonadal and perirenal fat, whereas we could not detect a signal in BAT or any other tissue. The absence of signal in brain, where Slc7a10 expression has been reported previously, could indicate that Slc7a10 protein levels in white adipose tissue are so much higher than in the brain that more tissue lysate would have been needed to detect a signal. Additionally, the brain could express an alternative splice product of Slc7a10, since immune reactive proteins are detected in the brain at ˜70 kDa and ˜43 kDa (FIG. 3b).

### Example 2

### Slc7a10/Asc-1 Regulation During Weight Gain and Obesity

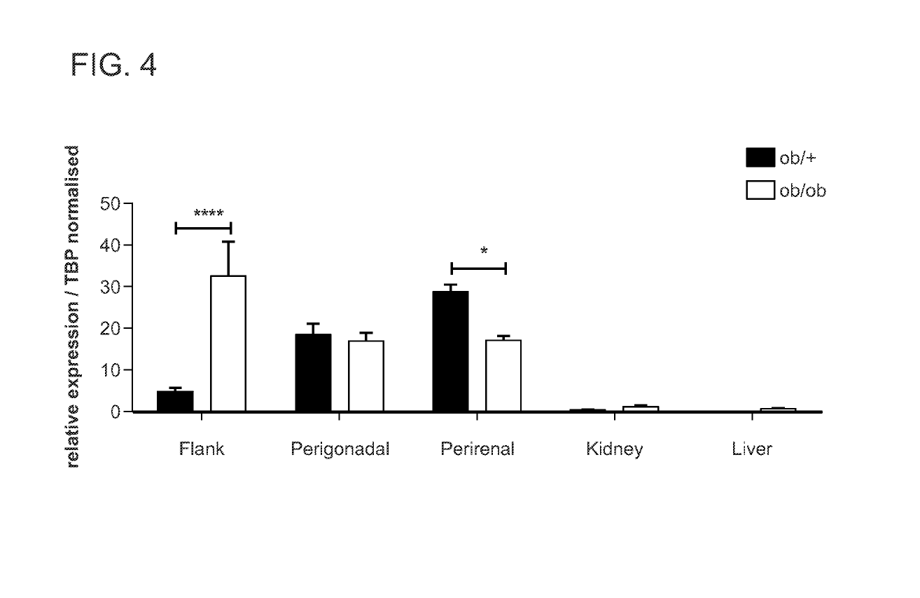

To gain insights into the regulation of Slc7a10/Asc-1 during weight gain and obesity Slc7a10/Asc-1 expression was compared in flank, perigonadal and perirenal fat as well as kidney and liver of ob/ob and control mice (FIG. 4). Significantly increased expression of Slc7a10/Asc-1 in flank fat and a significantly reduced expression in perirenal fat was observed whereas expression remained unchanged in perigonadal fat as well as kidney and liver. Importantly, however, for the use of Slc7a10/Asc-1 as a surface maker for white adipocytes, the overall high expression of Slc7a10/Asc-1 in adipocytes in comparison to other tissues remained unaltered.

### Example 3

### Slc7a10/Asc-1 Expression was then Investigated During Adipocyte Differentiation

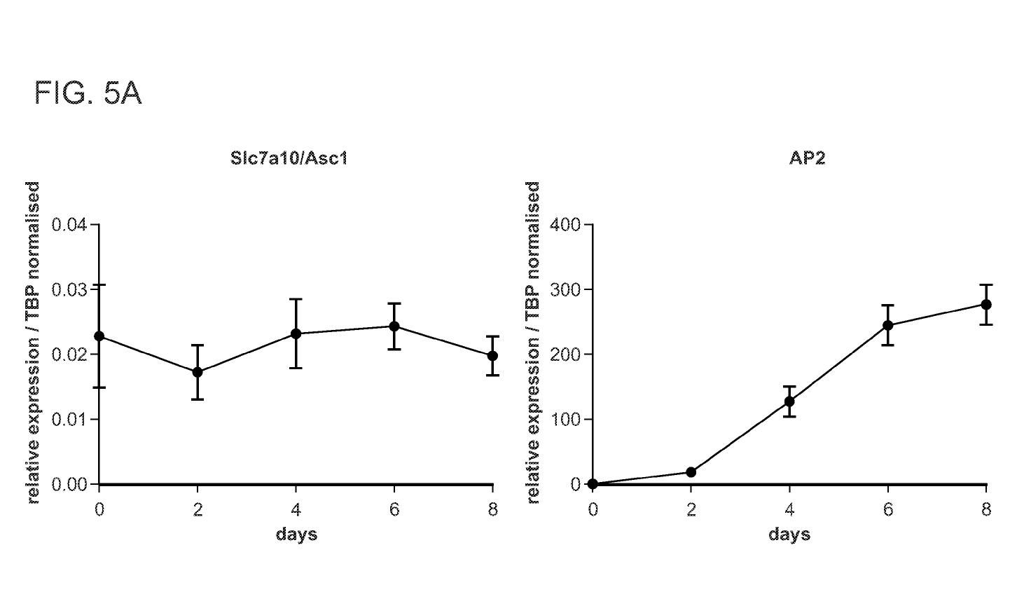

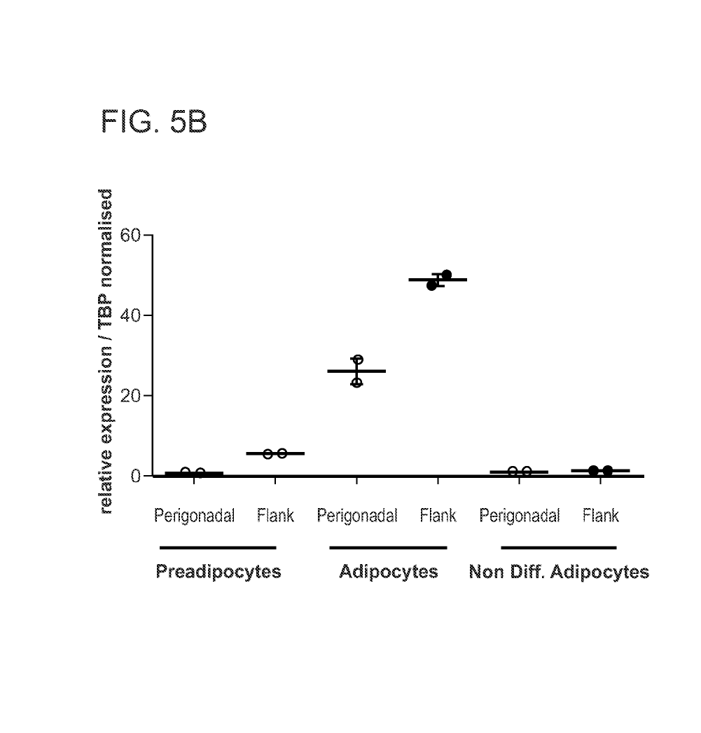

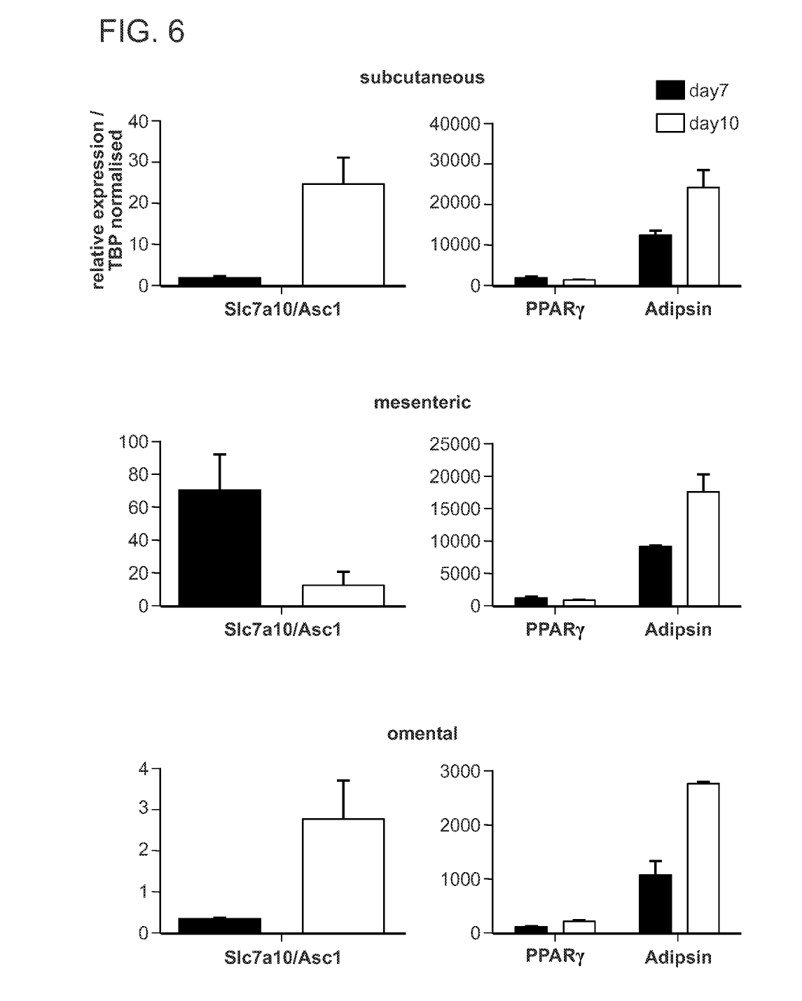

Slc7a10/Asc-1 expression was then investigated during adipocyte differentiation. Using 3T3-L1 cells. No significant changes in expression were detected during an eight day time course of differentiation, in sharp contrast to aP2 which increases progressively during adipocyte differentiation (FIG. 5a). Importantly, however, primary preadipocytes freshly isolated from perigonadal and flank fat showed a clear induction of Slc7a10/Asc-1 expression upon differentiation (FIG. 5b). Cells that failed to differentiate upon induction with the “differentiation cocktail” (as disclosed by Gesta, et al., Proc Natl Acad Sci 103: 6676-6681 (2006)) did not show an increased Slc7a10 expression. This indicates that the increased expression was intrinsic to adipocytes and not a direct consequence of the differentiation cocktail used. These data further support our previous results that Slc7a10/Asc-1 is a marker for mature white adipocytes.

### Example 4

### Slc7a10/Asc-1 Expression was Compared from In Vitro Cultured and Differentiated Primary Human Preadipocytes and Adipocytes

To extend this observation from mice to men, Slc7a10/Asc-1 expression was compared from in vitro cultured and differentiated primary human preadipocytes and adipocytes (FIG. 6). As observed during murine adipocyte differentiation, a strong increase in Slc7a10/Asc-1 expression was detected when day 7 immature human adipocytes were compared to day 10 fully mature subcutaneous and omental adipocytes. Interestingly, while the highest expression of Slc7a10/Asc-1 could be detected in mesenteric adipocytes, expression decreased from day 7 to day 10. Taken together it was shown that Slc7a10 is highly expressed in mature white adipocytes and not only could be used to identify adipocytes in vivo but also could be used to distinguish between white and brown adipocytes.

### Example 5

### Identification of Brown Adipocyte Specific Surface Proteins

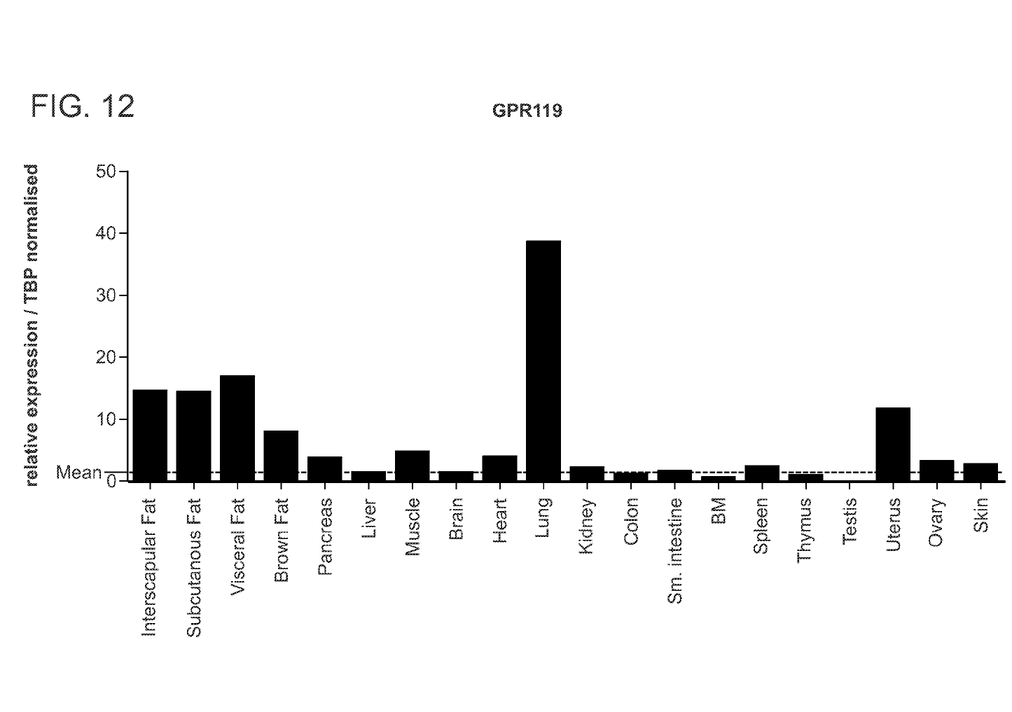

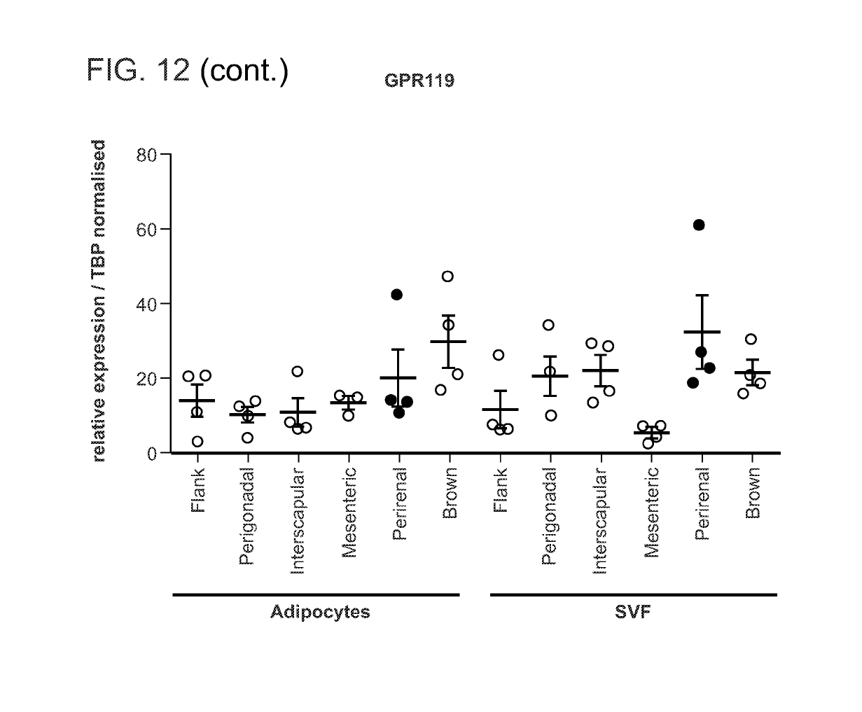

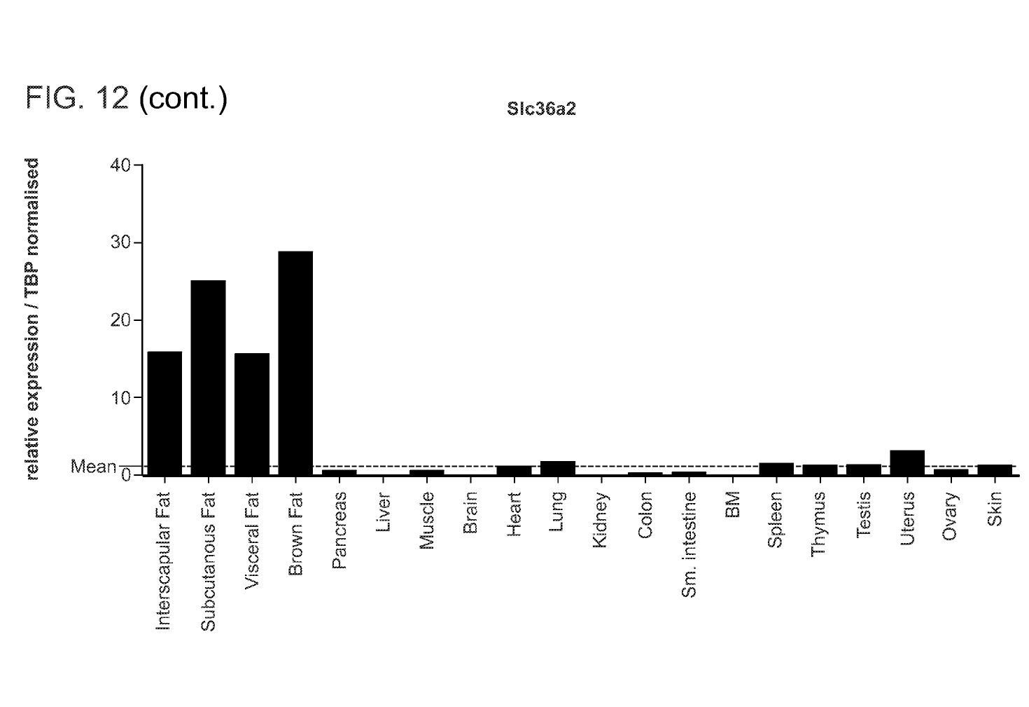

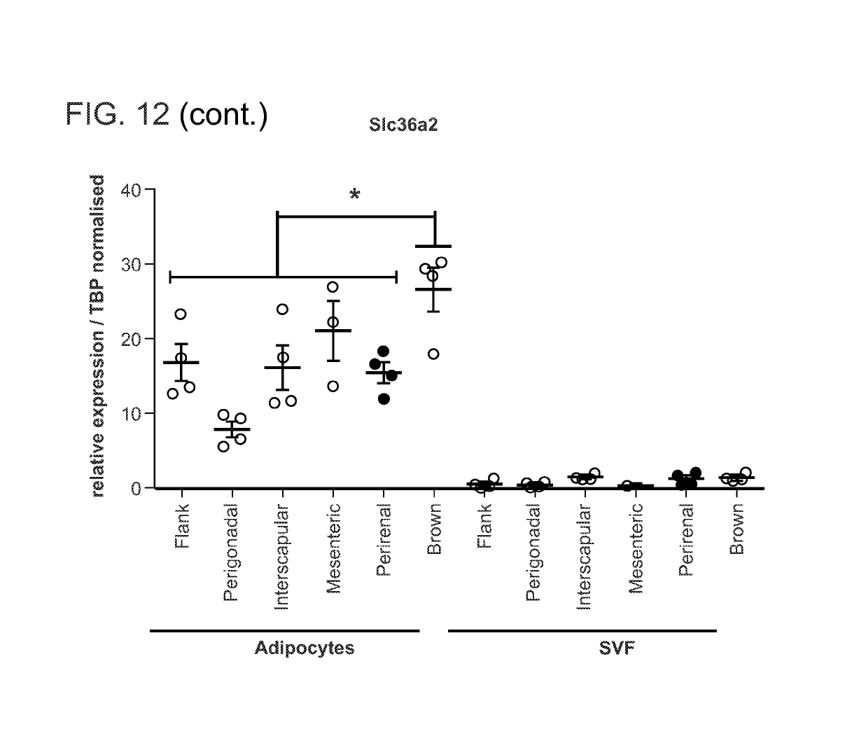

To identify brown adipocyte specific surface proteins, the same database search was performed as indicated in FIG. 1a but instead uncoupling protein 1 (UCP1) was used as a reference gene. Two potential brown fat specific transmembrane proteins (GPR119 and Slc36a2/PAT2) were identified. As shown in FIG. 12, GPR119 is widely expressed and its expression similar in adipocytes and the SVF. In contrast Slc36a2/PAT2 had highest expression in adipose tissue and was expressed in brown adipocytes at a significantly higher level than in comparison to white adipocytes and the SVF from all adipose tissues. Therefore, Slc36a2/PAT2 would fulfill the requirements of a brown adipocyte marker.

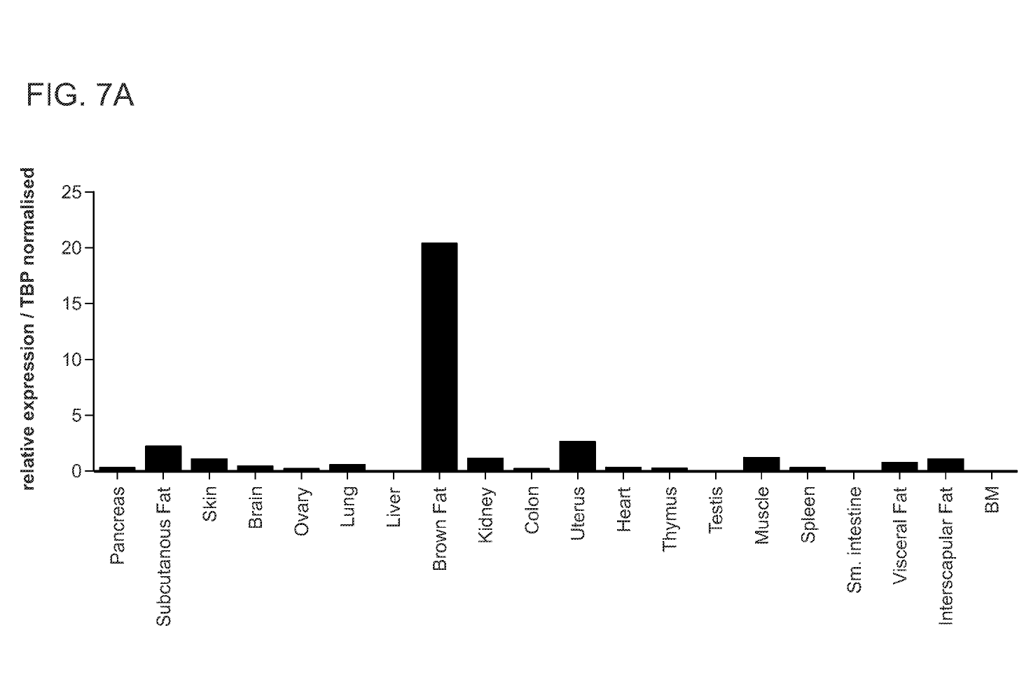

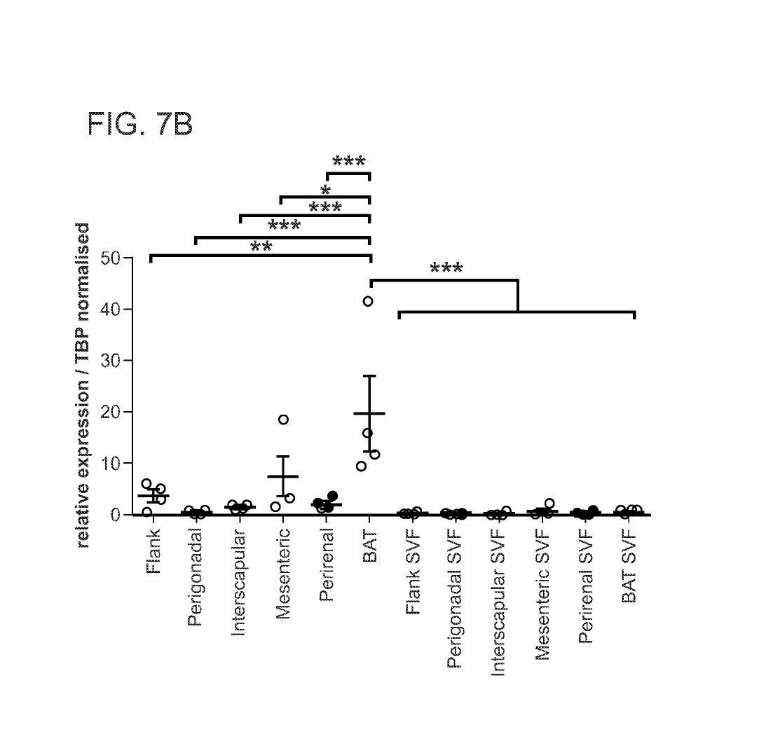

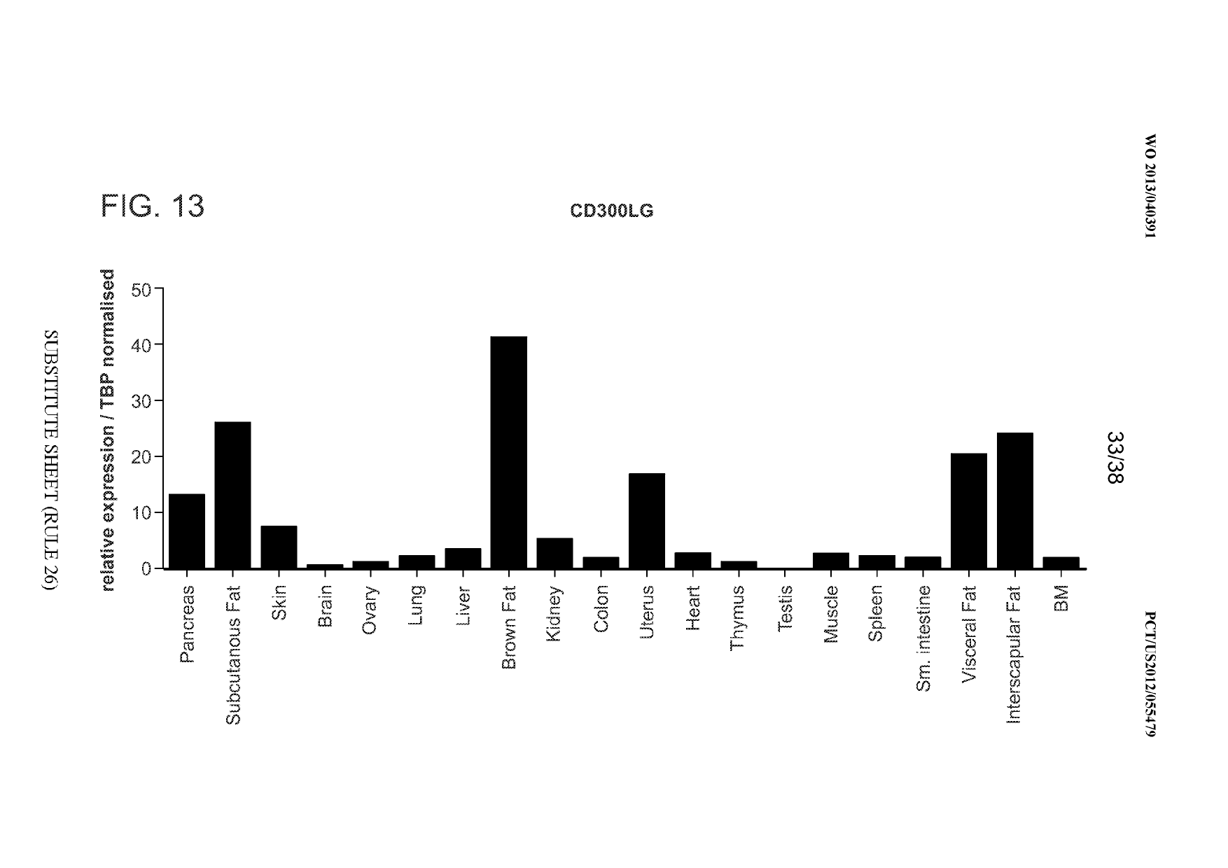

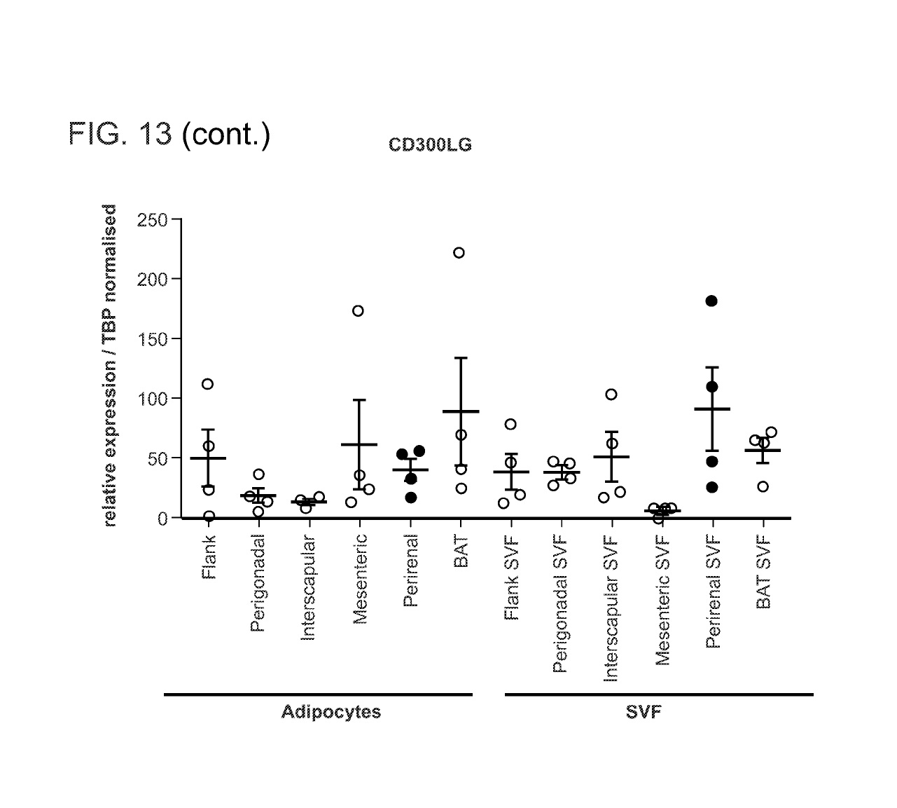

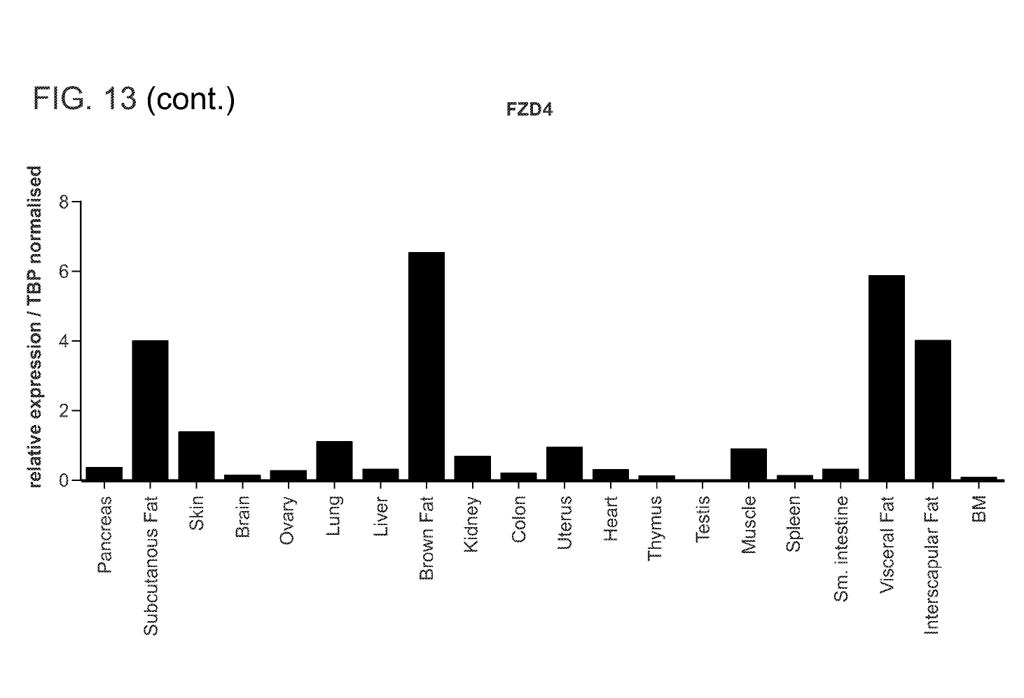

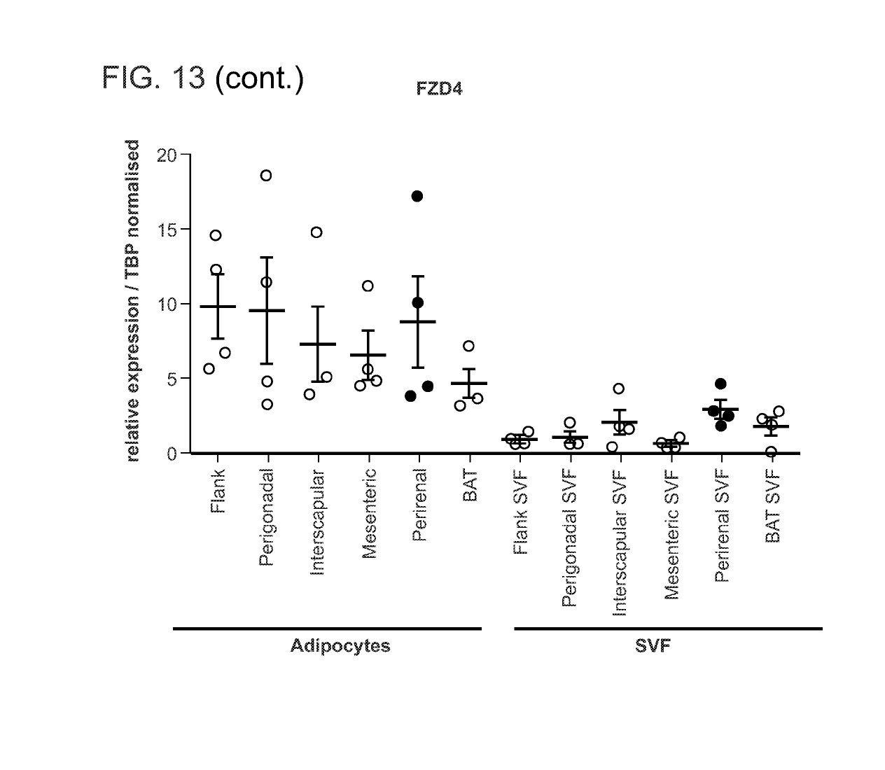

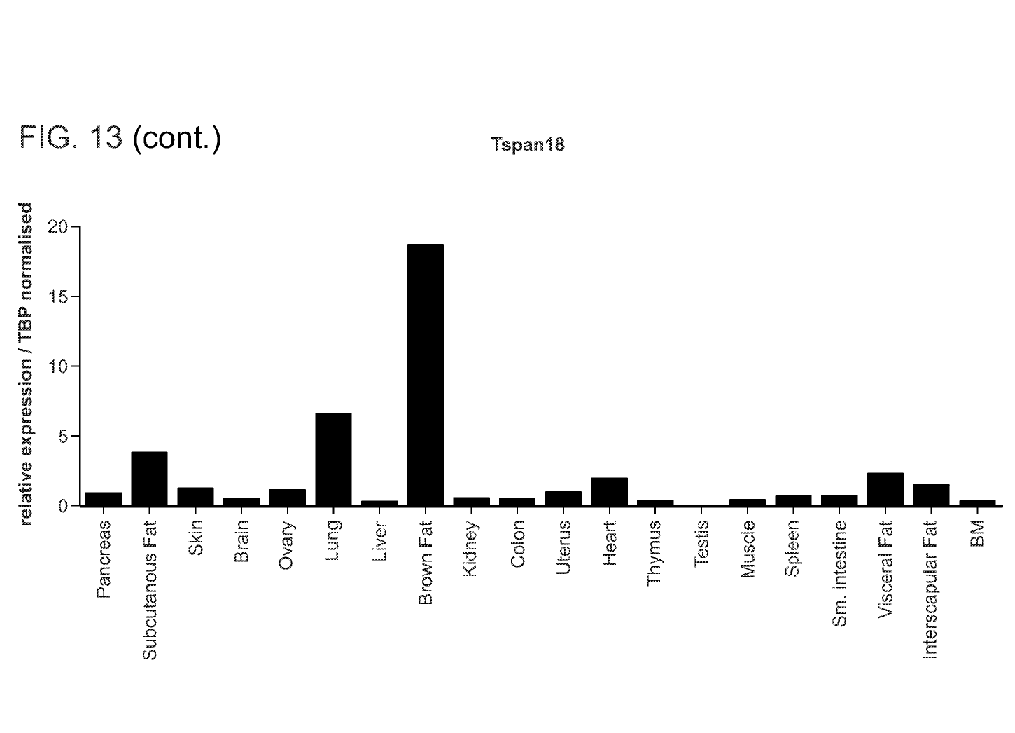

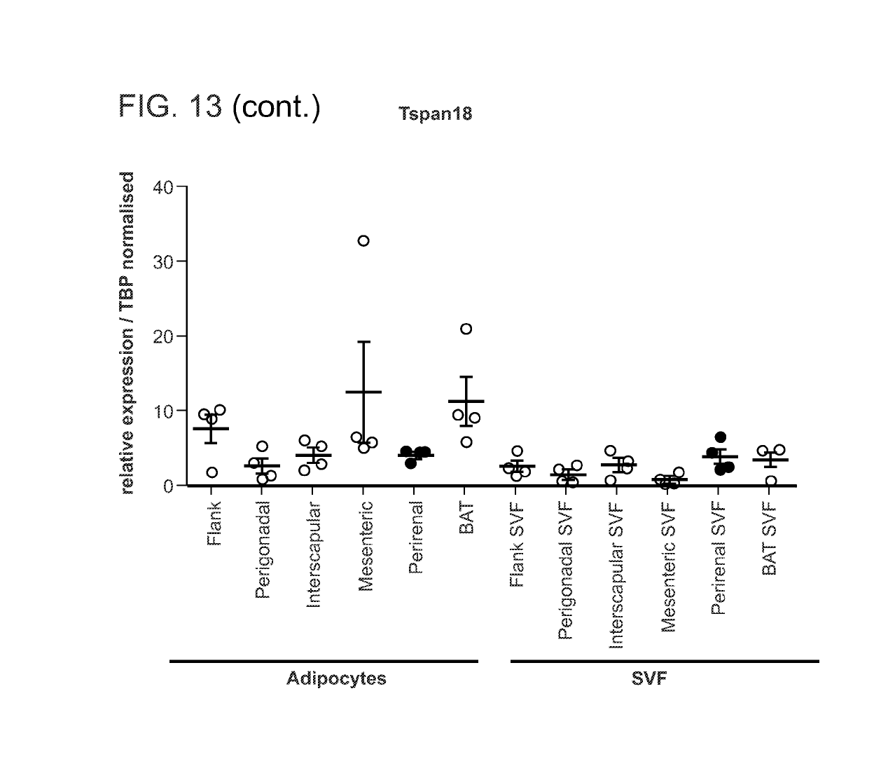

SLC36a2/PAT2 was then used as a reference gene for a final database search. Four transmembrane receptors were identified that correlated with Slc36a2/PAT2: CD300LG, Tspan18, FZD4 and P2X5 (FIG. 7 and FIG. 13). Like GPR119, CD300LG, Tspan18 and FZD4 showed a broad tissue distribution and none of these three receptors was expressed at significantly higher levels in brown adipocytes in comparison to white adipocytes and the SVF (FIG. 13). In contrast, P2X5 expression was ˜10 fold higher in BAT than in other tissues (FIG. 7a). Furthermore, P2X5 was significantly higher in expression in isolated brown adipocytes compared to white adipocytes and the corresponding SVFs (FIG. 7b). Therefore, P2X5 would fulfill the requirements of a brown adipocyte marker. Isolation of brown adipocytes is technically challenging and some contamination with surrounding white adipocytes could occur. Therefore, it is of special importance to notice that P2X5 expression in the interscapular adipocytes, surrounding the BAT was ˜10 fold lower than in the brown adipocytes.

Although the present inventors do not wish to be limited by theory, P2X5 (purinergic receptor P2X, ligand-gated ion channel, 5) is part of a seven member family of ATP gated ion channels (Garcia-Guzman, et al., FEBS Lett 388:123-127 (1996); North, Physiol Rev 82:1013-1067 (2002)). P2X5, like its family members, is a two transmembrane protein that forms homotrimeric or, together with P2RX1, heterotrimeric functional complexes (North, Physiol Rev 82:1013-1067 (2002)). The vast majority of humans have a T/G SNP at the 3′ splice site of exon 10 resulting in skipping of this exon. The lack of exon 10 results in the loss of the second transmembrane domain as well as parts of the ATP binding domain leading to a nonfunctional receptor (Kotnis, et al., Mol Pharmacol 77:953-960 (2010)). Expression of P2X5 has been reported in brain, heart, spinal cord and adrenal gland (Garcia-Guzman, et al., FEBS Lett 388:123-127 (1996)) and in the testes, lung, skin and skeletal muscle (Cox, et al. Gene 270:145-152, (2001)) of mice. Both groups detected strong signals in heart using Northern Blots whereas in the present invention comparably low levels were detected using Real-Time PCR. Interestingly, the expression pattern of hP2X5 (Le, et al., FEBS Lett. 418: 195-199 (1997)) is very similar to the expression pattern observed in the present invention. As for Slc7a10 discussed above, neither of the previous studies included adipose tissue.

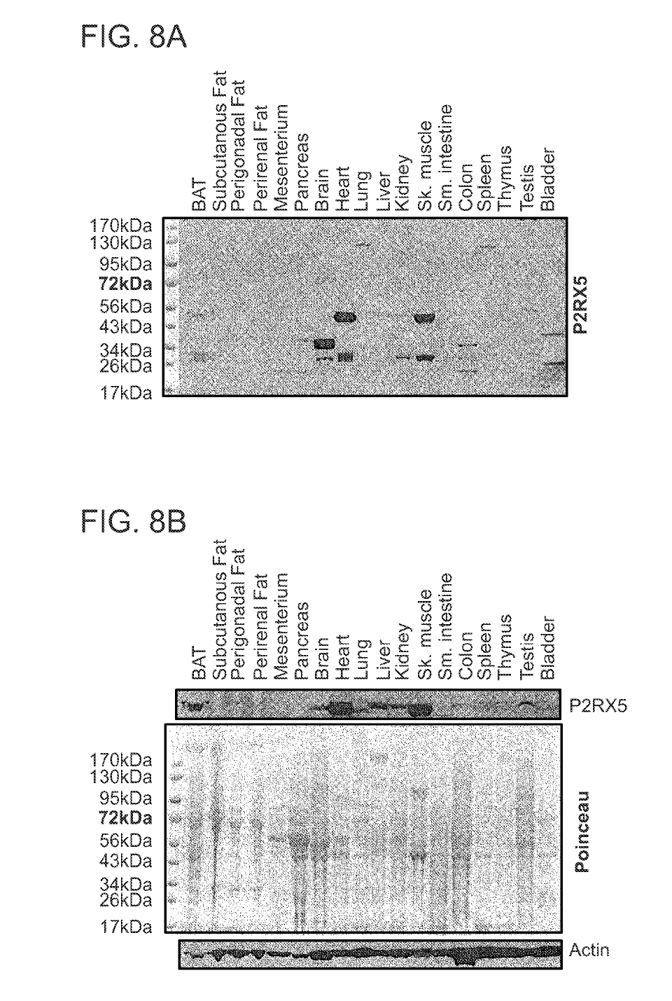

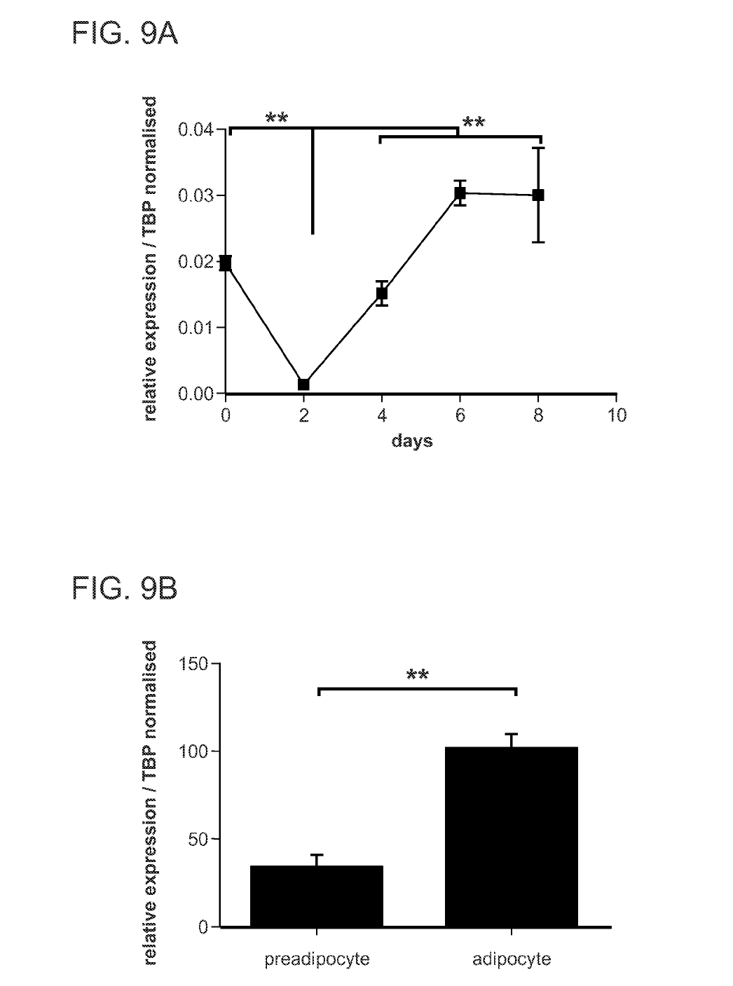

P2X5 has a predicted molecular weight of 51 kDa. Western Blots for P2X5 using a commercially available antibody showed multiple bands at different molecular weights (FIG. 8a). A strong signal at 51 kDa was detected in BAT but not in WAT tissues (FIG. 8b). However even stronger signals were observed by the inventors in heart liver and skeletal muscle, tissues that showed much lower mRNA expression levels. This discrepancy between mRNA and protein data indicate a potential cross reactivity of the antibody and needs to be addressed using different P2X5 antibodies. Finally, P2X5 expression during adipocyte differentiation of 3T3-L1 and brown preadipocytes was tested. Overall expression in 3T3-L1 was very low and while there was a significant drop in expression 48 hours after induction, there were no significant differences between preadipocytes and adipocytes (FIG. 9a). In contrast, P2X5 expression in brown preadipocytes appeared much higher and was further increased upon differentiation (FIG. 9b). Interestingly, Slc7a10 was not detected in either brown preadipocytes or brown adipocytes, further confirming the specificity of these surface proteins (data not shown).

### EQUIVALENTS

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure.

What is claimed is:

1. A composition comprising a P2X5 targeting agent coupled to a BAT agent, wherein the BAT agent is an agent that modifies brown adipose tissue (BAT) or brown adipocyte development.

2. A composition according to claim 1 wherein the BAT agent is an agent that increases BAT mass and or promotes BAT activity.

3. The composition of claim 1, wherein the BAT agent is selected from the group consisting of a bone morphogenetic protein (BMP) polypeptide, or fragment thereof, selected from the group consisting of BMP-2, BMP-4, BMP-6, BMP-7; fibroblast growth factors, selected from the group consisting of FGF-6, FGF-9; thiazolidinediones and small interfering RNAs.

4. The composition of claim 1, wherein the P2X5 targeting agent is selected from the group consisting of antibodies, antibody fragments and aptamers.

5. A method of targeting an agent to a brown adipose tissue or brown adipocyte in a subject, comprising administering to the subject a therapeutically effective amount of an agent to be targeted to a brown adipose tissue or brown adipocyte, wherein the agent is coupled to a P2X5 targeting agent.

6. (canceled)

7. (canceled)

8. A composition comprising a Slc36a2 targeting agent coupled to a BAT agent, wherein the BAT agent is an agent that modifies brown adipose tissue (BAT) or brown adipocyte development.

9. A composition according to claim 8 wherein the BAT agent is an agent that increases BAT mass and or promotes BAT activity.

10. The composition of claim 8, wherein the BAT agent is selected from the group consisting of a bone morphogenetic protein (BMP) polypeptide, or fragment thereof, selected from the group consisting of BMP-2, BMP-4, BMP-6, BMP-7; fibroblast growth factors, selected from the group consisting of FGF-6, FGF-9; thiazolidinediones and small interfering RNAs.

11. The composition of claim 8, wherein the Slc36a2 targeting agent is selected from the group consisting of antibodies, antibody fragments and aptamers.

12. A method of targeting an agent to a brown adipose tissue or brown adipocyte in a subject, comprising administering to the subject a therapeutically effective amount of an agent to be targeted to a brown adipose tissue or brown adipocyte, wherein the agent is coupled to a Slc36a2 targeting agent.

13. (canceled)

14. (canceled)

15. A composition comprising a Slc7a10 targeting agent coupled to a WAT agent, wherein the WAT agent is an agent that modifies white adipose tissue or white adipocyte development.

16. A composition according to claim 15, wherein the WAT agent is an agent that increases or decrease lipid storage and or increases or decreases adipokine secretion.

17. The composition of claim 15, wherein the WAT agent is selected from the group consisting of insulin, BMPs, FGFs, thiazolidinediones, small interfering RNAs, resistin, RBP4, TNFa, IL-6, and toxins.

18. The composition of claim 15, wherein the Slc7a10 targeting agent is selected from the group consisting of antibodies, antibody fragments and aptamers.

19. A method of targeting an agent to a white adipose tissue or white adipocyte in a subject, comprising administering to the subject a therapeutically effective amount of an agent to be targeted to a white adipose tissue or white adipocyte, wherein the agent is coupled to a Slc7a10 targeting agent.

20. (canceled)

21. (canceled)


SEQUENCE LISTING (39 sequences)

SEQ ID NO: 1 - protein, 483 aa, Homo sapiens
MSVTKSTEGPQGAVAIKLDLMSPPESAKKLENKDSTFLDESPSESAGLKKTKGITVFQALIHLVKGNMGTGILGLPLAVKNAGILMGPLSLLVMGFIACHCMHILVKCAQRFCKRLNKPFMDYGDTVMHGLEANPNAWLQNHAHWGRHIVSFFLIITQLGFCCVYIVFLADNLKQVVEAVNSTTNNCYSNETVILTPTMDSRLYMLSFLPFLVLLVLIRNLRILTIFSMLANISMLVSLVIIIQYITQEIPDPSRLPLVASWKTYPLFFGTAIFSFESIGVVLPLENKMKNARHFPAILSLGMSIVTSLYIGMAALGYLRFGDDIKASISLNLPNCWLYQSVKLLYIAGILCTYALQFYVPAEIIIPFAISRVSTRWALPLDLSIRLVMVCLTCLLAILIPRLDLVISLVGSVSGTALALIIPPLLEVTTFYSEGMSPLTIFKDALISILGFVGFVVGTYQALDELLKSEDSHPFSNSTTFVR

SEQ ID NO: 2 - protein, 523 aa, Homo sapiens
MAGHTQQPSGRGNPRPAPSPSPVPGTVPGASERVALKKEIGLLSACTIIIGNIIGSGIFISPKGVLEHSGSVGLALFVWVLGGGVTALGSLCYAELGVAIPKSGGDYAYVTEIFGGLAGFLLLWSAVLIMYPTSLAVISMTFSNYVLQPVFPNCIPPTTASRVLSMACLMLLTWVNSSSVRWATRIQDMFTGGKLLALSLIIGVGLLQIFQGHFEELRPSNAFAFWMTPSVGHLALAFLQGSFAFSGWNFLNYVTEEMVDARKNLPRAIFISIPLVTFVYTFTNIAYFTAMSPQELLSSNAVAVTFGEKLLGYFSWVMPVSVALSTFGGINGYLFTYSRLCFSGAREGHLPSLLAMIHVRHCTPIPALLVCCGATAVIMLVGDTYTLINYVSFINYLCYGVTILGLLLLRWRRPALHRPIKVNLLIPVAYLVFWAFLLVFSFISEPMVCGVGVIIILTGVPIFFLGVFWRSKPKCVHRLTESMTHWGQELCFVVYPQDAPEEEENGPCPPSLLPATDKPSKPQ

SEQ ID NO: 3 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 4 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 5 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 6 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 7 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 8 - protein, 208 aa, Homo sapiens
MALGQKLFITMSRGAGRLQGTLWALVFLGILVGMVVPSPAGTRANNTLLDSRGWGTLLSRSRAGLAGEIAGVNWESGYLVGIKRQRRLYCNVGIGFHLQVLPDGRISGTHEENPYSLLEISTVERGVVSLFGVRSALFVAMNSKGRLYATPSFQEECKFRETLLPNNYNAYESDLYQGTYIALSKYGRVKRGSKVSPIMTVTHFLPRI

SEQ ID NO: 9 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 10 - DNA, 20 bp, Artificial Sequence
  Description of Artificial Sequence Synthetic primer
ccttcccagaaaggaaaagg

SEQ ID NO: 11 - DNA, 20 bp, Artificial Sequence
  Description of Artificial Sequence Synthetic primer
caggagtcaacaggatgcaa

SEQ ID NO: 12 - DNA, 20 bp, Artificial Sequence
  Description of Artificial Sequence Synthetic primer
gacacgagctgcagacagac

SEQ ID NO: 13 - DNA, 20 bp, Artificial Sequence
  Description of Artificial Sequence Synthetic primer
tggcacataaaccgaacaaa

SEQ ID NO: 14 - DNA, 19 bp, Artificial Sequence
  Description of Artificial Sequence Synthetic primer
ctgcagctcaccatcctgt

SEQ ID NO: 15 - DNA, 20 bp, Artificial Sequence
  Description of Artificial Sequence Synthetic primer
cactctgcagggaagtgtca

SEQ ID NO: 16 - DNA, 20 bp, Artificial Sequence
  Description of Artificial Sequence Synthetic primer
gaggcctctggtcctgctat

SEQ ID NO: 17 - DNA, 20 bp, Artificial Sequence
  Description of Artificial Sequence Synthetic primer
acggtgtcaccttcaaatcc

SEQ ID NO: 18 - DNA, 20 bp, Artificial Sequence
  Description of Artificial Sequence Synthetic primer
ttctgcgtatcgtctgcaag

SEQ ID NO: 19 - DNA, 20 bp, Artificial Sequence
  Description of Artificial Sequence Synthetic primer
gctctagccgaacacgaatc

SEQ ID NO: 20 - DNA, 20 bp, Artificial Sequence
  Description of Artificial Sequence Synthetic primer
tgaaacagcagacagggaca

SEQ ID NO: 21 - DNA, 20 bp, Artificial Sequence
  Description of Artificial Sequence Synthetic primer
ggcgtcctgtcttgacactc

SEQ ID NO: 22 - DNA, 20 bp, Artificial Sequence
  Description of Artificial Sequence Synthetic primer
gcgagagtttctggcagagt

SEQ ID NO: 23 - DNA, 20 bp, Artificial Sequence
  Description of Artificial Sequence Synthetic primer
ccgagatagctgccagagtt

SEQ ID NO: 24 - DNA, 20 bp, Artificial Sequence
  Description of Artificial Sequence Synthetic primer
ggaccgaagctgacattttg

SEQ ID NO: 25 - DNA, 20 bp, Artificial Sequence
  Description of Artificial Sequence Synthetic primer
tcctcagaccactggtagcc

SEQ ID NO: 26 - DNA, 19 bp, Artificial Sequence
  Description of Artificial Sequence Synthetic primer
gtgccaagaagctgcagag

SEQ ID NO: 27 - DNA, 20 bp, Artificial Sequence
  Description of Artificial Sequence Synthetic primer
tgttgcctttgaccagatga

SEQ ID NO: 28 - DNA, 20 bp, Artificial Sequence
  Description of Artificial Sequence Synthetic primer
gcatggttgcttcttccttc

SEQ ID NO: 29 - DNA, 20 bp, Artificial Sequence
  Description of Artificial Sequence Synthetic primer
gagactcgagctgcagacct

SEQ ID NO: 30 - DNA, 20 bp, Artificial Sequence
  Description of Artificial Sequence Synthetic primer
gcggtcctcctcactcttac

SEQ ID NO: 31 - DNA, 20 bp, Artificial Sequence
  Description of Artificial Sequence Synthetic primer
caggctgatctcacactgga

SEQ ID NO: 32 - DNA, 20 bp, Artificial Sequence
  Description of Artificial Sequence Synthetic primer
ccctgagcttcacctacctg

SEQ ID NO: 33 - DNA, 20 bp, Artificial Sequence
  Description of Artificial Sequence Synthetic primer
ttcaggctgcagattgtgag

SEQ ID NO: 34 - DNA, 20 bp, Artificial Sequence
  Description of Artificial Sequence Synthetic primer
gggtggcactcaagaaagag

SEQ ID NO: 35 - DNA, 20 bp, Artificial Sequence
  Description of Artificial Sequence Synthetic primer
agtgttccaggacacccttg

SEQ ID NO: 36 - DNA, 20 bp, Artificial Sequence
  Description of Artificial Sequence Synthetic primer
ctggagggagaatttcagga

SEQ ID NO: 37 - DNA, 20 bp, Artificial Sequence
  Description of Artificial Sequence Synthetic primer
gctcctccagcttctgctta

SEQ ID NO: 38 - DNA, 20 bp, Artificial Sequence
  Description of Artificial Sequence Synthetic primer
gtggcgctcaagaaggagat

SEQ ID NO: 39 - DNA, 19 bp, Artificial Sequence
  Description of Artificial Sequence Synthetic primer
ccttgggcgagatgaagat